(19) United States
(12) Patent Application Publication
(10) Pub. No.: US 2022/0204950 A1
(43) Pub. Date: Jun. 30, 2022

(54) A NEW TYPE OF ENZYME COMPOSITION

(71) Applicant: Belief BioMed Limited, Hong Kong (CN)

(72) Inventors: Cheng CHENG, Hong Kong (CN); Jing ZHENG, Hong Kong (CN)

(21) Appl. No.: 17/604,995

(22) PCT Filed: Apr. 17, 2020
(86) PCT No.: PCT/CN2020/085366

§ 371 Date: Oct. 19, 2021

(30) Foreign Application Priority Data
Apr. 19, 2019 (CN) 201910322504.8

(51) Int. Cl. C12N 9/02; A61P 25/16; C12N 15/86; C12N 9/88

(52) U.S. Cl. CPC C12N 9/0071; C12Y 114/16002; A61P 25/16; A61K 38/00; C12N 9/88; C12Y 401/01028; C12N 2750/14143; C12N 15/86

(57) ABSTRACT

The present disclosure relates to a tyrosine hydroxylase (TH) variant lacking 60 to 120 amino acid residues at the N terminus, and a pharmaceutical composition comprising the TH variant lacking 60 to 120 amino acid residues at the N terminus and the aromatic L-amino acid decarboxylase (AADC). The present disclosure further relates to a nucleotide construct, a vector plasmid, a cell or a virus comprising the TH variant or the pharmaceutical composition, as well as use of the virus in the manufacture of a medicament for treating neurodegenerative diseases (e.g., Parkinson's Disease).

### FIELD OF THE INVENTION

The present disclosure relates to a pharmaceutical composition comprising a tyrosine hydroxylase variant and an aromatic L-amino acid decarboxylase. The present disclosure further relates to a nucleotide construct comprising a polynucleotide encoding the tyrosine hydroxylase variant or a polynucleotide encoding the aforementioned composition, a vector comprising the nucleotide construct, a cell prepared by transfection with the vector, and a virus comprising the aforementioned nucleotide construct. The present disclosure further relates to use of the virus in the manufacture of a medicament for treating neurodegenerative diseases (e.g., Parkinson's disease, PD), which belongs to the field of genetic engineering technology.

### BACKGROUND

Parkinson's Disease (PD) is a severe neurodegenerative disease characterized by main symptoms including tremor, rigidity and dyskinesia. The pathological hallmark of PD is the progressive degradation of dopaminergic neurons in the substantia nigra (SN) of the brain, which leads to impaired innervation of dopaminergic neurons and a reduction in dopamine concentration in this striatum. Consequently, pharmacological methods that can increase dopaminergic delivery to the striatum are effective therapeutic intervenes for PD. Dopamine replacement therapy (i.e. oral levodopa, L-Dopa) is the primary pharmaceutical treatment for PD at present. Although this therapy significantly improves the life quality of PD patients in the short term, the effectiveness of dopamine replacement therapy will gradually decrease over time. After more than 5 to 10 years, almost all PD patients will finally progress to conditions that can hardly be treated by oral L-Dopa.

The purpose of enzyme replacement therapies is to compensate for the decrease in dopamine synthesis and secretion caused by dopaminergic neuron degeneration in SN. The mechanism underlying this therapeutic method is the delivery of genes encoding enzymes necessary for dopamine synthesis into GABAergic neurons in striatum, which leads to sustaining de novo synthesis of dopamine in these neurons and release of the synthesized dopamine into striatum. This therapy can improve dyskinesia and restrict the side effects caused by elevated levels of dopamine outside the basal ganglia. However, increasing dopamine concentration will negatively regulate the activity of tyrosine hydroxylase (TH), thereby limiting the ability of ectopic dopamine synthesis by TH.

Consequently, there is substantial need to find an enzyme replacement therapy with better therapeutic effect for PD treating.

### SUMMARY OF THE INVENTION

In one aspect, the present disclosure provides a tyrosine hydroxylase variant comprising an amino acid sequence set forth in SEQ ID NO: 1 except for an N-terminal deletion of 60 to 120 amino acid residues, or a fragment, a derivative or an analog thereof having at least 80% sequence identity.

In certain embodiments, the tyrosine hydroxylase variant comprises an amino acid sequence set forth in SEQ ID NO: 1 except for an N-terminal deletion of 80 to 100 amino acid residues, or a fragment, a derivative or an analog thereof having at least 80% sequence identity.

In certain embodiments, the tyrosine hydroxylase variant comprises an amino acid sequence set forth in SEQ ID NO: 1 except for an N-terminal deletion of 80 to 90 amino acid residues, or a fragment, a derivative or an analog thereof having at least 80% sequence identity.

In certain embodiments, the tyrosine hydroxylase variant comprises an amino acid sequence set forth in SEQ ID NO: 2 or a fragment, a derivative or an analog thereof having at least 80% sequence identity.

In certain embodiments, the tyrosine hydroxylase variant further comprises a tag protein attached to N terminus or C terminus.

In certain embodiments, the tag protein is HA, Myc or Flag.

In certain embodiments, the tyrosine hydroxylase variant comprises an amino acid sequence set forth in SEQ ID NO: 3.

In another aspect, the present disclosure provides a composition, comprising the tyrosine hydroxylase variant mentioned above.

In certain embodiments, the composition further comprises an aromatic L-amino acid decarboxylase.

In certain embodiments, the aromatic L-amino acid decarboxylase comprises an amino acid sequence set forth in any of SEQ ID NOs: 4-9 or a fragment, a derivative or an analog thereof having at least 80% sequence identity.

In certain embodiments, the aromatic L-amino acid decarboxylase further comprises a tag protein attached to the N terminus or the C terminus.

In certain embodiments, the tag protein is HA, Myc or Flag.

In certain embodiments, the aromatic L-amino acid decarboxylase has an amino acid sequence set forth in SEQ ID NO: 10.

In another aspect, the present disclosure provides a polynucleotide construct, comprising a first polynucleotide encoding the tyrosine hydroxylase variant mentioned above, and/or a second polynucleotide encoding the aromatic L-amino acid decarboxylase as defined above.

In certain embodiments, the first polynucleotide has a nucleotide sequence set forth in SEQ ID NO: 12 or 13, or has a nucleotide sequence having at least 80% sequence identity to SEQ ID NO: 12 or 13.

In certain embodiments, the second polynucleotide has a nucleotide sequence set forth in any of SEQ ID NOs: 14-21, or has a nucleotide sequence having at least 80% sequence identity to any of SEQ ID NOs: 14-21.

In certain embodiments, the polynucleotide construct further comprises a promoter operably linked to the first polynucleotide and/or to the second polynucleotide.

In certain embodiments, the promoter comprises a neuron-specific promoter.

In another aspect, the present disclosure provides a vector, comprising the polynucleotide construct mentioned above.

In certain embodiments, the first polynucleotide and the second polynucleotide are constructed in one vector, or in different vectors.

In certain embodiments, the first polynucleotide and the second polynucleotide are constructed in one vector, and the vector further comprises a third polynucleotide inserted between the first polynucleotide and the second polynucleotide.

In certain embodiments, the third polynucleotide encodes a self-cleavable sequence and/or an internal ribosome entry site (IRES).

In certain embodiments, the vector is selected from the group consisting of herpes simplex virus vector, adenovirus vector, and adeno-associated virus vector.

In certain embodiments, the vector comprises a plasmid.

In another aspect, the present disclosure provides a host cell comprising or transfected by the vector mentioned above.

In another aspect, the present disclosure provides a virus comprising a virus genome, wherein the virus genome comprises the polynucleotide construct mentioned above or comprises a nucleic acid expressed from the polynucleotide construct mentioned above.

In another aspect, the present disclosure provides a pharmaceutical composition, comprising the virus mentioned above and a pharmaceutically acceptable carrier.

In another aspect, the present disclosure provides use of the tyrosine hydroxylase variant mentioned above, the composition mentioned above, the nucleotide construct mentioned above, the vector mentioned above, the host cell mentioned above, the virus mentioned above, or the pharmaceutical composition mentioned above, in the manufacture of a medicament for treating a neurodegenerative disease in a subject.

In certain embodiments, the neurodegenerative disease is Parkinson's disease.

In certain embodiments, the subject is a mammal, preferably a human, a rat, or a mouse.

In another aspect, the present disclosure provides a method of treating a neurodegenerative disease in a subject in need thereof, comprising administering to the subject a therapeutically effective amount of the tyrosine hydroxylase variant mentioned above, the composition mentioned above, the nucleotide construct mentioned above, the vector mentioned above, the virus mentioned above, or the pharmaceutical mentioned above.

In certain embodiments, the neurodegenerative disease is Parkinson's disease.

In certain embodiments, the subject is a mammal, preferably a human, a rat, or a mouse.

The present disclosure further provides the following embodiments:

### Embodiment 1

A tyrosine hydroxylase variant comprising a tyrosine hydroxylase having an amino acid sequence set forth in SEQ ID NO: 1 but lacking 60 to 120 amino acid residues at N terminus.

### Embodiment 2

The tyrosine hydroxylase variant of embodiment 1, comprising a tyrosine hydroxylase having an amino acid sequence set forth in SEQ ID NO: 1 but lacking 80 to 100 amino acid residues at N terminus.

### Embodiment 3

The tyrosine hydroxylase variant of embodiment 2, comprising a tyrosine hydroxylase having an amino acid sequence set forth in SEQ ID NO: 1 but lacking 80 to 90 amino acid residues at N terminus.

### Embodiment 4

The tyrosine hydroxylase variant of embodiment 2 or 3, wherein the tyrosine hydroxylase variant comprises a protein having an amino acid sequence set forth in SEQ ID NO: 2, or a tyrosine hydroxylase derivative having 80% sequence identity to the amino acid sequence set forth in SEQ ID NO: 2, preferably, the tyrosine hydroxylase variant optionally further comprises a tag protein at the N terminus or the C terminus, and more preferably, said tag protein is HA, Myc or Flag.

### Embodiment 5

The tyrosine hydroxylase variant of embodiment 4, comprising a protein having an amino acid sequence set forth in SEQ ID NO: 3.

### Embodiment 6

A composition, comprising at least one tyrosine hydroxylase variant of any of embodiments 1 to 5.

### Embodiment 7

The composition of embodiment 6, further comprises aromatic L-amino acid decarboxylase.

### Embodiment 8

The composition of embodiment 7, wherein said aromatic L-amino acid decarboxylase is a full-length aromatic L-amino acid decarboxylase, which comprises a protein having an amino acid sequence set forth in any of SEQ ID NOs: 4-9 or an aromatic L-amino acid decarboxylase derivative having 80% sequence identity with the amino acid sequence set forth in any of SEQ ID NOs: 4-9, preferably, said aromatic L-amino acid decarboxylase optionally further comprises a tag protein at the N terminus or the C terminus, and more preferably, said tag protein is HA, Myc or Flag.

### Embodiment 9

The composition of embodiment 8, wherein the aromatic L-amino acid decarboxylase has an amino acid sequence set forth in SEQ ID NO: 10.

### Embodiment 10

A nucleotide construct, comprising a polynucleotide encoding the tyrosine hydroxylase variant of any of embodiments 1-5, or a polynucleotide encoding the composition of any of embodiments 6-9.

### Embodiment 11

The nucleotide construct of embodiment 10, wherein the polynucleotide encoding the tyrosine hydroxylase variant has a nucleotide sequence that is set forth in SEQ ID NO: 12 or 13, or that has more than 80% identity to SEQ ID NO: 12 or 13, and/or the polynucleotide encoding the aromatic L-amino acid decarboxylase has a nucleotide sequence that is set forth in any of SEQ ID NOs: 14-21, or that has more than 80% identity to any of SEQ ID NOs: 14-21.

### Embodiment 12

A vector plasmid, comprising the nucleotide construct of embodiment 10 or 11.

### Embodiment 13

The vector plasmid of embodiment 12, wherein the polynucleotide encoding the tyrosine hydroxylase variant and the polynucleotide encoding the aromatic L-amino acid decarboxylase are constructed in one vector plasmid, or in different vector plasmids.

### Embodiment 14

The vector plasmid of embodiment 12, wherein the vector plasmid is selected from the group consisting of herpes simplex virus vector plasmid, adenovirus vector plasmid, and adeno-associated virus vector plasmid.

### Embodiment 15

A cell, wherein the cell is prepared by transfection with the vector plasmid of any of embodiments 12-14.

### Embodiment 16

A virus comprising the nucleotide construct of embodiment 10 or 11 as genome thereof.

### Embodiment 17

A pharmaceutical composition, comprising the virus of embodiment 16 and a pharmaceutically acceptable carrier.

### Embodiment 18

Use of the tyrosine hydroxylase variant of any of embodiments 1-5, the pharmaceutical composition of any of embodiments 6-9, the nucleotide construct of embodiment 10 or 11, the vector plasmid of any of embodiments 12-14, the cell of embodiment 15, the virus of embodiment 16, or the pharmaceutical composition of embodiment 17, in the manufacture of a medicament for treating neurodegenerative diseases in a subject.

### Embodiment 19

The use of embodiment 18, wherein the neurodegenerative disease is Parkinson's disease.

### Embodiment 20

The use of embodiment 18, wherein the subject is a mammal, preferably a human, a rat, or a mouse.

### BRIEF DESCRIPTION OF THE FIGURES

FIG. 1 shows the construction of a recombinant AAV vector carrying an expression cassette that comprises the human synapsin promoter, the polynucleotide encoding the HA-tagged variant of human tyrosine hydroxylase (TH), a T2A peptide and the Myc-tagged human aromatic L-amino acid decarboxylase (AADC), the WPRE sequence and the human growth hormone (hGH) poly(A) signal according to certain embodiments of the present disclosure.

FIG. 2 shows the statistical quantification graph of expression of a series of enzyme compositions comprising the THs with deletions at N terminus and the full-length AADC for promoting dopamine de novo synthesis in the 293 cell line, as measured by high performance liquid chromatography (HPLC). GFP indicates a negative control; WT indicates the full-length or wild-type TH in the dual-enzyme composition; Isob indicates another isoform of the TH; 40 indicates a TH with 40 amino acids deleted at N terminus; 60 indicates a TH with 60 amino acids deleted at N terminus; 80 indicates a TH with 80 amino acids deleted at N terminus; 90 indicates a TH with 90 amino acids deleted at N terminus; 100 indicates a TH with 100 amino acids deleted at N terminus; 120 indicates a TH with 120 amino acids deleted at N terminus; 150 indicates a TH with 150 amino acids deleted at N terminus; 164 indicates a TH with 164 amino acids deleted at N terminus; and 190 indicates a TH with 190 amino acids deleted at N terminus. Error bars represent SEM. Ns, not significant. *p<0.05 and ****p<0.0001, one-way ANOVA.

FIG. 3 shows the representative immunohistochemistry images of anti-TH staining in the substantia nigra/ventral tegmental area (SNNTA) region (upper panel) and in caudate-putamen (CP) region (lower panel) of brain slices in a unilaterally 6-OHDA-lesioned mouse successfully modeling PD symptoms. The right side was 6-OHDA lesioned, and the left was control side. Scale bar, 1 mm.

FIG. 4 shows the schematic illustration of time course for stereotaxic surgeries and apomorphine rotation tests (FIG. 4a). Two weeks after the unilateral 6-OHDA lesion, mice were screened for apomorphine-induced significant motor asymmetry which was represented as contralateral rotation. Both groups showed statistically equivalent rotation frequency, which is calculated as net turns (ipsilateral to contralateral) per minute. Two weeks later, screened animals received intrastriatal injections of viral vectors expressing the composition comprising the human TH variant with a deletion of 90 amino acids at N terminus and the full-length human AADC (TH90del/AADC). The GFP-expressing virus was injected as a control. Four weeks after viral injections, apomorphine-induced rotation tests were performed again to assess the functional benefit of our treatments. FIG. 4b shows the significant behavioral recovery in the group with TH90del/AADC viral injections. Wherein, the pound sign indicates a significant recovery from the motor asymmetry phenotype in the group injected with the TH90del/AADC virus compared to the control group (GFP). The asterisk sign indicates a significant recovery from the motor asymmetry phenotype after viral injection in the TH90del/AADC group. Error bars represent SEM. ###p<0.001 and ****p<0.0001, Student's t test.

### DETAILED DESCRIPTION

The present disclosure is further described below through specific embodiments and experimental data. Although specific terms are used below for the purpose of clarity, these terms are not meant to define or limit the scope of the present disclosure.

As used herein, “a,” “an,” or “the” can mean one or more than one. For example, “a” cell can mean a single cell or a plurality of cells.

As used herein, unless specifically indicated otherwise, the word “or” is used in the inclusive sense of “and/or” and not the exclusive sense of “either/or.”

Unless specifically indicated otherwise, the number range described herein can include each number within the range and each subrange.

All publications, patent applications, patents, and other references mentioned herein are incorporated by reference herein in their entirety.

The present disclosure provides a tyrosine hydroxylase variant.

In one aspect, the present disclosure provides tyrosine hydroxylase (TH) variants comprising an amino acid sequence set forth in SEQ ID NO: 1 except for an N-terminal deletion of 60 to 120 amino acid residues. In other words, the TH variants provided herein are N-terminal deletion variants of the full-length TH having an amino acid sequence of SEQ ID NO: 1. The TH variant lacks from 60 to 120 amino acid residues at the N-terminus of the amino acid sequence of SEQ ID NO: 1.

In certain embodiments, the N-terminal deletion has a length ranging from 60 to 120, 70 to 120, 80 to 120, 90 to 120, 100 to 120, 60 to 110, 60 to 100, 60 to 90, 70 to 110, 80 to 100, or 80 to 90 amino acid residues. In certain embodiments, the N-terminal deletion has a length of 80, 81, 82, 83, 84, 85, 86, 87, 88, 89, 90, 91, 92, 93, 94, 95, 96, 97, 98, 99 or 100 amino acids. In certain embodiments, the N-terminal deletion starts from the 1^{st} amino acid residue of SEQ ID NO: 1, in other words, an N-terminal deletion of 60 amino acid residues means the deletion from the 1^{st} to the 60^{th} amino acid residue from SEQ ID NO: 1.

In certain embodiments, the N-terminal deletion variant of TH is a bioactive fragment of TH. As used herein, the term “bioactive fragment” refers to a polypeptide fragment of a specific protein that can retain entire or at least partial functions of the specific protein. Generally, a bioactive fragment of TH retains at least 50% biological activity, preferably 60%, 70%, 80%, 90%, 95%, 99%, or 100% biological activity of TH.

The present disclosure provides a tyrosine hydroxylase (also referred to as TH) variant, wherein the TH variant is a human TH having an amino acid sequence set forth in SEQ ID NO: 1 except for an N-terminal deletion of 60 to 120 amino acid residues. Preferably the TH variant is a human TH having an amino acid sequence set forth in SEQ ID NO: 1 except for an N-terminal deletion of 80 to 100 amino acid residues. More preferably, the TH variant is a human TH having an amino acid sequence set forth in SEQ ID NO: 1 except for an N-terminal deletion of 80 to 90 amino acid residues, e.g. a human TH having an N-terminal deletion of 80, 81, 82, 83, 84, 85, 86, 87, 88, 89, 90, 91, 92, 93, 94, 95, 96, 97, 98, 99, or 100 amino acid residues in SEQ ID NO: 1.

In one embodiment, the TH variant is a TH with an N-terminal deletion of 90 amino acid residues. In one embodiment, the TH variant comprises a protein having an amino acid sequence set forth in SEQ ID NO: 2.

The present disclosure also provides fragments, derivatives or analogs of the TH variants provided herein, and such fragments, derivatives or analogs substantially maintain the biological function or activity of the TH variants. The term “fragment” with respect to a polypeptide or polynucleotide sequence means a portion of that sequence. The term “derivatives” or “analogs”, with respect to the polypeptides provided herein (for example the TH variants and AADC provided herein), include but is not limited to, (i) a counterpart polypeptide with one or more conservative or non-conservative amino acid residue substitution (preferably conservative amino acid residue substitution), or (ii) a counterpart polypeptide in which one or more amino acid residues have a substituted group, or (iii) a counterpart polypeptide in which the polypeptide is fused with or attached to another compound (e.g., a compound that extends the half-life of the polypeptide, such as polyethylene glycol), or (iv) a counterpart polypeptide formed by fusion of the polypeptide to an appended amino acid sequence (e.g., a leader sequence, a secretion sequence, a sequence used for purifying this polypeptide, a proteinogen sequence, or a fusion protein). These fragments, derivatives and analogs as defined herein are within the scope known by those skilled in the art.

In one embodiment, the fragments, derivatives or analogs of the TH variants comprise an amino acid sequence having at least 80% (e.g. at least 80%, 90%, 95%, or 99%) sequence identity to the amino acid sequence set forth in SEQ ID NO: 1. “Percent (%) sequence identity” is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the reference sequence. Conservative substitution of the amino acid residues is not considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

In certain embodiments, the fragments, derivatives or analogs of the TH variants provided herein comprise an amino acid sequence having at least 80% (e.g. at least 80%, 90%, 95%, or 99%) sequence identity to the amino acid sequence set forth in SEQ ID NO: 2.

In certain embodiments, the fragment, derivative, or analog of a TH variant is formed by substitution, deletion, or addition of one or a few (e.g. 2, 3, 4, 5, 6, 7, 8, 9, or 10) amino acid residues in the amino acid sequence set forth in SEQ ID NO: 1 or SEQ ID NO: 2. In certain embodiments, the fragment, derivative, or analog of a TH variant functions as the protein having an amino acid sequence set forth in SEQ ID NO: 1 or SEQ ID NO: 2. The TH variant and the fragment, derivative, or analog thereof have at least 50% (e.g. at least 60%, 70%, 80%, 85%, 90%, 95%, 99%) activity of the protein having an amino acid sequence set forth in SEQ ID NO: 1 or SEQ ID NO: 2.

In certain embodiments, the TH variant may optionally further comprise a tag protein. As used herein, the terms “tag protein” and “protein tag” are interchangeable, and refer to a polypeptide or protein that is fused with a target protein by in vitro DNA recombination technology to facilitate expression, detection, tracing, or purification of the target protein. Protein tags include, but are not limited to, His6, Flag, GST, MBP, HA, GFP, or Myc. In certain embodiments, the tag protein includes, without limitation, HA, Myc, or Flag. In certain embodiments, HA comprises an amino acid sequence of SEQ ID NO: 22. In certain embodiments, Myc comprises an amino acid sequence of SEQ ID NO: 24. In certain embodiments, Flag comprises an amino acid sequence of SEQ ID NO: 26. The tag protein can be attached to the N terminus or C terminus of the TH variants or the fragments, derivatives, or analogs thereof. In certain embodiments, the TH variants provided herein comprise an amino acid sequence of SEQ ID NO: 3, or a fragment, derivative, or analog thereof having at least 80% sequence identity to SEQ ID NO: 3.

In another aspect, the present disclosure also provides a composition, comprising the TH variant as described above, or a fragment, a derivative or an analog thereof.

In one embodiment, the composition further comprises an aromatic L-amino acid decarboxylase (AADC), for example, a full-length AADC, or a fragment, a derivative, or an analog of the full-length AADC. In one embodiment, the full-length AADC comprises the protein having an amino acid sequence set forth in any of SEQ ID NOs: 4-9. In one embodiment, the fragment, derivative, or analog of the full-length AADC has at least 80% (e.g. at least 80%, 90%, 95%, 99%) sequence identity to the amino acid sequence set forth in any of SEQ ID NOs: 4-9. In one embodiment, the fragment, derivative, or analog of the full-length AADC is formed by substitution, deletion, or addition of one or a limited number of (e.g. 2, 3, 4, 5, 6, 7, 8, 9, or 10) amino acid residues in the amino acid sequence set forth in any of SEQ ID NOs: 4-9. Those skilled in the art would understand that the fragments, derivatives and analogs of the full-length AADC substantially retain the biological function or activity of the full-length AADC. In one embodiment, the fragment, derivative, or analog of the full-length AADC functions as the protein having the amino acid sequence set forth in any of SEQ ID NOs: 4-9. The fragment, derivative, or analog of the full-length AADC has at least 50% (e.g. at least 60%, 70%, 80%, 85%, 90%, 95%, 99%) activity of the protein having an amino acid sequence set forth in any of SEQ ID NOs: 4-9.

In certain embodiments, the AADC may optionally further comprise a tag protein at N terminus or C terminus, which preferably includes, but is not limited to, HA, Myc, or Flag. The tag protein can be attached to the N terminus or C terminus of the AADC. In one particular embodiment, the AADC has an amino acid sequence set forth in SEQ ID NO: 10, or has an amino acid sequence having at least 80% sequence identity to SEQ ID NO: 10.

In one particular embodiment, the AADC may be any of the natural isoforms encoded by DDC gene or the variant thereof. Some alternatively spliced transcriptional variants encoding different AADC isoforms have been identified in the art. Specifically, the DDC gene produces 7 different transcriptional variants, which encode 6 different protein isoforms. Both variants 1 and 2 transcribed from DDC gene encode AADC isoform 1. In a preferred embodiment, the full-length AADC is AADC isoform 1 (NCBI reference sequence: NP_000781.1), encoded by a polynucleotide that is the coding region of transcriptional variant 1 or 2 of DDC gene. Those skilled in the art, based on the prior art, can reasonably expect that all these naturally-existing isoforms (e.g., the AADC having the amino acid sequence set forth in any of SEQ ID NOs: 4-9) are applicable in the present disclosure and may achieve identical or similar effect.

In certain embodiments, the composition provided herein is a pharmaceutical composition. In certain embodiments, the composition provided herein further comprises a pharmaceutically acceptable carrier. In certain embodiments, the composition provided herein is for therapeutic use.

In certain embodiments, the composition provided herein is an enzyme composition. As used herein, the terms “enzyme composition” refer to the composition comprising an AADC and a TH variant with an N-terminal deletion of more than 60 and less than 120 amino acid residues (e.g., 80, 90 or 100 amino acid residues). In one embodiment, the amino acid sequence of the TH variant with an N-terminal deletion of 90 amino acid residues is set forth in SEQ ID NO: 2. AADC can be a full-length AADC, whose amino acid sequence is set forth in SEQ ID NO: 4. In view of the teachings of the present disclosure and the prior art, those skilled in the art would further understand that the TH variant with an N-terminal deletion of 90 amino acid residues or the full-length AADC as used in the present disclosure, would also include variation forms thereof, and such variation forms have the same or similar functions as those of the TH with an N-terminal deletion of 90 amino acid residues or the full-length AADC, despite of having a few differences in the amino acid sequence. These variation forms include, but are not limited to, deletions, insertions, and/or substitutions of one or more (e.g., one to five) amino acid residues, and addition of one or more (usually within 20, preferably within 10, and more preferably within 5) amino acid residues at C terminus and/or N terminus. It is well known to those skilled in the art that substitution with amino acid residues having similar or close properties, for example, substitution between isoleucine and leucine, would not change functions of the resultant protein. As another example, appending a tag at C terminus and/or N terminus that comprises one or more amino acids and is convenient for purification or detection usually may not affect functions of the resultant protein. In one particular embodiment, the “enzyme composition” used in the present disclosure may comprise the N-terminally HA-tagged TH lacking 90 amino acids at N terminus and a full-length AADC with a Myc tag at C terminus.

Polynucleotide Construct

In another aspect, the present disclosure also provides a polynucleotide construct, comprising a polynucleotide encoding the TH variant, or a fragment, derivative or analog thereof. In certain embodiments, the polynucleotide construct further comprises a polynucleotide encoding the AADC or a derivative thereof. In certain embodiments, the present disclosure provides a polynucleotide construct encoding the pharmaceutical composition or the enzyme composition as described above.

As used herein, the term “polynucleotide” refers to a DNA molecule or an RNA molecule. The DNA molecule includes cDNA, genomic DNA, or synthetic DNA. The DNA molecule may be single-stranded or double-stranded. The sequence encoding for a mature polypeptide can be identical to the coding sequence of a particular protein or its degeneracy variant. A degeneracy variant refers to a polynucleotide sequence that encodes a protein but is different from the coding sequence of the protein by genetic code degeneracy.

In one embodiment, the polynucleotide encoding the TH variant has a nucleotide sequence that is set forth in SEQ ID NO: 12 or 13 or that has at least 80%, preferably at least 80%, 90%, 95%, 99% sequence identity to SEQ ID NO: 12 or 13, and/or the polynucleotide encoding the AADC has a nucleotide sequence that is set forth in any of SEQ ID NOs: 14-21 or that has at least 80%, preferably 80%, 90%, 95%, 99% or more sequence identity to any of SEQ ID NOs: 14-21. In one embodiment, the polynucleotide is a degeneracy variant of SEQ ID NO: 12 or 13, and encodes the same TH variant. In one embodiment, the polynucleotide is a degeneracy variant of one of SEQ ID NO: 14-21, and encodes the same AADC.

In certain embodiments, the polynucleotide encoding the fragment, derivative or analog of the TH variant has a nucleotide sequence that has at least 80%, preferably at least 80%, 90%, 95%, 99% identity to SEQ ID NO: 12 or 13. In certain embodiments, the polynucleotide encoding the fragment, derivative or analog of the AADC has a nucleotide sequence that has at least 80%, preferably at least 80%, 90%, 95%, 99% identity to any of SEQ ID NO: 14-21.

In one particular embodiment, the polynucleotide of the TH is a variant formed by substitution, deletion, or addition of one or a limited number of (e.g. 2, 3, 4, 5, 6, 7, 8, 9, or 10) nucleotide residues or codons in the nucleotide sequence set forth in SEQ ID NO: 12 or 13, and functions as polynucleotide set forth in SEQ ID NO: 12 or 13. This variant has at least 90% (e.g. at least 95%, 99%) sequence identity to or biological activity of the polynucleotide set forth in SEQ ID NO: 12 or 13.

In one particular embodiment, the polynucleotide encoding the AADC is a variant formed by substitution, deletion, or addition of one or a limited number of (e.g. 2, 3, 4, 5, 6, 7, 8, 9, 10) nucleotide residues or codons in the nucleotide sequences set forth in any of SEQ ID NOs: 14-21, and functions as the polynucleotide set forth in any of SEQ ID NOs: 14-21. This variant has at least 90% (e.g., at least 95%, 99%) sequence identity to or biological activity of any of SEQ ID NOs: 14-21.

In one particular embodiment, the polynucleotide construct further comprises a promoter. Those skilled in the art will recognize that the expression of an exogenous gene requires a proper promoter, including, but not limited to, a species-specific, inducible, tissue-specific, or cell cycle specific promoter. The precise regulation of gene expression usually depends on a promoter that guides the initiation of RNA transcription. The promoter may be either constitutive or inducible. The promoter may be expressed in all cell types (such as CMV) or in specific cell types. For central nervous system (CNS), neuron-specific promoters include, but are not limited to, neurofilament, synapsin, or serotonin receptor; glial-specific promoters include, but are not limited to, glial fibrillary acidic protein (GFAP), S100 or glutamine synthase. In particular embodiments, a human synapsin promoter is used for transcribing the polynucleotide in the vector plasmid of the present disclosure, and the protein encoded by the polynucleotide described above will be specifically expressed in neurons. Those skilled in the art can reasonably expect other neuron-specific promoters to have corresponding functions.

Vector

In another aspect, the disclosure provides a vector comprising the polynucleotide construct as described above.

In one embodiment, the TH variant is a human TH variant; the AADC is a human AADC.

In one embodiment, the polynucleotide encoding the TH variant (or a derivative thereof) and the polynucleotide encoding the AADC (or a derivative thereof) of the composition can be constructed in one vector plasmid or in different vector plasmids.

In one particular embodiment, the vector comprises three portions as shown below (from 5′ to 3′):

1) a polynucleotide encoding the TH variant provided herein or a derivative thereof;

2) a T2A sequence encoding a peptide capable of self-cleaving; and

3) a polynucleotide encoding the AADC variant provided herein or a derivative thereof.

In one particular embodiment, the vector comprises three portions as shown below (from 5′ to 3′):

1) a polynucleotide encoding TH with an N-terminal deletion of 90 amino acid residues;

2) a T2A sequence encoding a peptide capable of self-cleaving; and

3) a polynucleotide encoding full-length AADC.

In certain embodiments, the T2A sequence comprises a nucleotide sequence of SEQ ID NO: 28.

In one particular embodiment, when the polynucleotide encoding the TH with an N-terminal deletion of 90 amino acid residues and the polynucleotide encoding the full-length AADC are in the same vector plasmid, a T2A sequence encoding a peptide capable of self-cleaving is added between the two, thereby constructing a monocistron that expresses two proteins synchronously.

In another particular embodiment, an internal ribosome entry site (IRES) is added between the polynucleotide encoding the TH with an N-terminal deletion of 90 amino acid residues and the polynucleotide encoding the full-length AADC. When IRES nucleotide sequence is present downstream the stop codon of an mRNA, it can lead to the reentry of ribosomes, thereby initiating translation of a second Open Reading Frame (ORF).

In one particular embodiment, the polynucleotide encoding the TH with an N-terminal deletion of 90 amino acid residues and the polynucleotide encoding the AADC (e.g. full-length or fragment, derivative, or analog thereof) may also be constructed in different vectors, respectively.

As used herein, the term “vector” refers to a molecular tool that can transport and transduce exogenous target genes (e.g., the polynucleotide described in the present disclosure) into target cells. Examples of vectors include, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector can be a DNA vector, a RNA vector, a viral vector, a non-viral vector, a recombinant vector, or an expression vector. As used herein, the term “expression vector” refers to a vector that can allow expression of the exogenous target genes after being transported or transduced into target cells. An expression vector can provide appropriate nucleotide sequences which can initiate transcription in the target cell (i.e., promoters). As used herein, the term “viral vector” refers to an expression vector having viral sequence for example a viral terminal repeat sequence. Those skilled in the art would understand that it is a preferential way that exogenous target genes are transduced into and expressed in target cells by viral vectors in the field of gene therapy.

In one embodiment, the vector provided herein comprises a plasmid vector.

In one embodiment, the vector is a viral vector. In one embodiment, the vector is selected from the group consisting of herpes simplex virus (HSV) vector, adenovirus (Ad) vector, and adeno-associated virus (AAV) vector. In one embodiment, the vector is capable of being expressed in central nervous system. Effective expression vectors for the central nervous system (CNS) include, but are not limited to, HSV, Ad or AAV, preferably AAV.

In certain embodiments, the vector comprises or is an AAV vector. AAV is a single-stranded human DNA parvovirus whose genome has a size of about 4.7 kilobases (kb). The AAV genome contains two major genes: the rep gene, which encodes the rep proteins (Rep 76, Rep 68, Rep 52 and Rep 40) and the cap gene, which encodes AAV structural proteins (VP-1, VP-2 and VP-3), flanked by 5′ inverted terminal repeat (ITR) and 3′ ITR. The term “AAV vector” as used herein encompasses any vector (e.g. plasmid) that comprises one or more heterologous sequence flanked by at least one, or two AAV inverted terminal repeat sequences. The term “AAV ITR”, as well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity, allowing intra-strand base-pairing to occur within this portion of the ITR.

An AAV ITR can be derived from any AAV, including but not limited to AAV serotype 1 (AAV 1), AAV 2, AAV 3, AAV 4, AAV 5, AAV 6, AAV 7, AAV 8, AAV 9, AAV 10, AAV 11, AAV 12, avian AAV, bovine AAV, canine AAV, equine AAV, and ovine AAV and any other AAV now known or later discovered. For details please see, e.g., BERNARD N F et al., VIROLOGY, volume 2, chapter 69 (4th ed., Lippincott-Raven Publishers), Gao et al., (2004) J. Virol. 78:6381-6388. The nucleotide sequences of AAV ITR regions are known. See for example Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.). An early description of the AAV1, AAV2 and AAV3 terminal repeat sequences is provided by Xiao, X., (1996), “Characterization of adeno-associated virus (AAV) DNA replication and integration,” Ph.D. Dissertation, University of Pittsburgh, Pittsburgh, Pa. (incorporated herein to it its entirety).

An AAV ITR can be a native AAV ITR, or alternatively can be altered from a native AAV ITR, for example by mutation, deletion or insertion, so long as the altered ITR can still mediate the desired biological functions such as replication, virus packaging, integration, and the like. The 5′ and 3′ ITRs which flank a selected nucleotide sequence in an AAV vector need not necessarily be identical or derived from the same AAV serotype, so long as they function as intended, for example, to allow for excision and rescue of the sequence of interest from and integration into the recipient cell genome.

In certain embodiments, the AAV vector provided herein comprises an expression cassette having a size suitable for being packaged into an AAV virus particle. For example, the size of the expression cassette in the AAV vector can be up to the size limit of the genome size of the AAV to be used, for example, up to 5.2 kb. In certain embodiments, the expression cassette in the AAV vector has a size of no more than 5.2 kb, no more than about 5 kb, no more than about 4.5 kb, no more than about 4 kb, no more than about 3.5 kb, no more than about 3 kb, no more than about 2.5 kb, see for example, Dong, J. Y. et al. (Nov. 10, 1996). In certain embodiments, the AAV vector plasmid provided herein comprises a transgene expression cassette which is less than 5000 bp (e.g. about 4550 bp), and includes ITRs, a promoter, WPRE, and poly(A). The transgene comprises the nucleotide construct provided herein. In some embodiments, the AAV vectors can be recombinant. A recombinant AAV (rAAV) vector can comprise one or more heterologous sequences that is not of the same viral origin (e.g. from a non-AAV virus, or from a different serotype of AAV, or from a partially or completely synthetic sequence). In certain embodiments, the nucleotide construct provided herein is flanked by the at least one AAV ITR.

AAV vectors can be constructed using methods known in the art. General principles of rAAV vector construction are known in the art. See, e.g., Carter, 1992, Current Opinion in Biotechnology, 3:533-539; and Muzyczka, 1992, Curr. Top. Microbiol. Immunol., 158:97-129. For example, a heterologous sequence can be directly inserted between the ITRs of an AAV genome in which the Rep gene and/or Cap gene have been deleted. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same, and fused to 5′ and 3′ of a heterologous sequence using standard ligation techniques, such as those described in Sambrook et al., supra. AAV vectors which contain AAV ITRs are commercially available and have been described in, e.g., U.S. Pat. No. 5,139,941.

In one particular embodiment, the ectopic synthesis of dopamine and the expression of an enzyme composition are carried out by an AAV vector in the present disclosure. However, in view of the disclosures of the present disclosure and the prior art, those skilled in the art should further understand that the AAV vectors used in the present disclosure can also include variations thereof, which include but are not limited to DNA sequence variations that do not affect basic functions of AAV vectors, or the changes of AAV serotypes.

As used herein, the term “ectopic synthesis” or “de novo synthesis” refers to the initiation of certain compound production by utilizing some techniques in cells, tissues or organs that do not originally synthesize this compound. In one particular embodiment, the enzyme composition used in the present disclosure can function in medium spiny neurons (MSNs) that do not originally synthesize dopamine in striatum and promote synthesis and secretion of dopamine in this brain region, which can play important roles in relieving PD-related phenotypes.

Virus Particles

In another aspect, the present disclosure provides a cell prepared by transfection with the vector (e.g. plasmid or viral vector) as described above.

The present disclosure provides a virus particle comprising, as its genome, a nucleotide construct as described above.

The AAV virus particle can be produced from the AAV vector described above. AAV particles can be produced by introducing an AAV vector provided herein into a suitable host cell using known techniques, such as by transfection, together with other necessary machineries such as plasmids encoding AAV cap/rep gene, and helper genes provided by either adeno or herpes viruses (see, for example, M. F. Naso et al, BioDrugs, 31(4): 317-334 (2017), which are incorporated herein to its entirety). The AAV vector can be expressed in the host cell and packaged into virus particles.

The AAV virus particle provided herein has a capsid protein which is encoded by a cap gene. In some embodiments, the capsid protein can be native or recombinant. In some embodiments, the capsid protein can be modified or chimeric or synthetic. A modified capsid can comprise modifications such as insertions, additions, deletions, or mutations. For example, a modified capsid may incorporate a detection or purification tag. A chimeric capsid comprises portions of two or more capsid sequences. A synthetic capsid comprise synthetic or artificially designed sequence. The capsid structure of AAV is also known in the art and described in more detail in Bernard N F et al., supra.

In some embodiments, the cap gene or the capsid protein is derived from two or more AAV serotypes. As used herein, the term “serotype” with respect to an AAV refers to the capsid protein reactivity with defined antisera. It is known in the art that various AAV serotypes are functionally and structurally related, even at the genetic level (see; e.g., Blacklow, pp. 165-174 of “Parvoviruses and Human Disease” J. R. Pattison, ed. (1988); and Rose, Comprehensive Virology 3:1, 1974). However, AAV virus particles of different serotypes may have different tissue tropisms (see, for details, in, Nonnenmacher M et al., Gene Ther., 2012 June; 19(6): 649-658), and can be selected as appropriate for gene therapy for a target tissue. In some embodiments, the cap gene or the capsid protein can have a specific tropism profile. The term “tropism profile” refers to the pattern of transduction of one or more target cells, tissues and/or organs. For example, the capsid protein may have a tropism profile specific for brain, liver (e.g. hepatocytes), eye, muscle, lung, kidney, intestine, pancreas, salivary gland, or synovia, or any other suitable cells, tissue or organs.

In some embodiments, the cap gene or the capsid protein is derived from any suitable AAV capsid gene or protein, for example, without limitation, AAV capsid gene or protein derived from AAV1, AAV2, AAV3, AAV4, AAV5, AAV6, AAV7, AAV8, AAV9, AAV12, AAV843, AAVbb2, AAVcyS, AAVrh10, AAVrh20, AAVrh39, AAVrh43, AAVrh64, AAVhu37, AAV3B, AAVhu48, AAVhu43, AAVhu44, AAVhu46, AAVhu19, AAVhu20, AAVhu23, AAVhu22, AAVhu24, AAVhu21, AAVhu27, AAVhu28, AAVhu29, AAVhu63, AAVhu64, AAVhu13, AAVhu56, AAVhu57, AAVhu49, AAVhu58, AAVhu34, AAVhu45, AAVhu47, AAVhu51, AAVhu52, AAVhu T41, AAVhu S17, AAVhu T88, AAVhu T71, AAVhu T70, AAVhu T40, AAVhu T32, AAVhu T17, AAVhu LG15, AAVhu9, AAVhu10, AAVhu11, AAVhu53, AAVhu55, AAVhu54, AAVhu7, AAVhu18, AAVhu15, AAVhu16, AAVhu25, AAVhu60, AAVch5, AAVhu3, AAVhu1, AAVhu4, AAVhu2, AAVhu61, AAVrh62, AAVrh48, AAVrh54, AAVrh55, AAVcy2, AAVrh35, AAVrh37, AAVrh36, AAVcy6, AAVcy4, AAVcy3, AAVcy5, AAVrh13, AAVrh38, AAVhu66, AAVhu42, AAVhu67, AAVhu40, AAVhu41, AAVrh40, AAVrh2, AAVbb1, AAVhu17, AAVhu6, AAVrh25, AAVpi2, AAVpi3, AAVrh57, AAVrh50, AAVrh49, AAVhu39, AAVrh58, AAVrh61, AAVrh52, AAVrh53, AAVrh51, AAVhu14, AAVhu31, AAVhu32, AAVrh34, AAVrh33, AAVrh32, Avian AAV ATCC VR-865, Avian AAV strain DA-1 or Bovine AAV. The capsid of AAV843 is the identical to the synthetic capsid AAVXL32 as disclosed in WO2019241324A1 (incorporated herein to its entirety), and AAV843 is also disclosed in for example, Xu J. et al., Int J Clin Exp Med, 2019; 12(8):10253-10261.

More examples of AAV capsid gene sequences and protein sequences can be found in GenBank database, see, GenBank Accession Nos: AF043303, AF028705, AF028704, J02275, J01901, J02275, X01457, AF288061, AH009962, AY028226, AY028223, NC 001358, NC 001540, AF513851, AF513852, AY530579, AY631965, AY631966; AF063497, AF085716, AF513852, AY530579, AAS99264.1, AY243022, AY243015, AY530560, AY530600, AY530611, AY530628, AY530553, AY530606, AY530583, AY530555, AY530607, AY530580, AY530569, NC 006263, NC 005889, NC 001862, AY530609, AY530581, AY530563, AY530591, AY530562, AY530584, AY530622, AY530601, AY530586, AY243021, AY530570, AY530589, AY530595, AY530572, AY530588, AY530575, AY530565, AY530590, AY530602, AY530566, AY530587, AY530585, AY530564, AY530592, AY530623, AY530574, AY530593, AY530560, AY530594, AY530573, AF513852, AY530624, AY530561, AY242997, AY530625, AY530567, AY530556, AY530578, AY530568, AY530618, AY243020, AY530579, AY530619, AY530596, AY530612, AY243000, AY530597, AY530620, AY242998, AY530598, AY242999, AY530599, AY243016, NC 001729, NC 001401, AY243018, NC 001863, AY530608, AY243019, NC 001829, AY530610, AY243017, AY243001, AY530613, AY243013, AY243002, AY530614, AY243003, AY695378, AY530558, AY530626, AY695376, AY695375, AY530605, AY695374, AY530603, AY530627, AY695373, AY695372, AY530604, AY695371, AY530600, AY695370, AY530559, AY695377, AY243007, AY243023, AY186198, AY629583, NC 004828, AY530629, AY530576, AY243015, AY388617, AY530577, AY530582, AY530615, AY530621, AY530617, AY530557, AY530616 or AY530554.

In certain embodiments, the AAV virus particle comprises a capsid protein derived from AAV9, and hence has a serotype of AAV9. The capsid gene sequence of AAV9 is known in the art, for example, from GenBank database, see, GenBank Accession No AY530579.

In certain embodiments, the AAV virus particle comprises a capsid protein from one AAV serotype and AAV ITRs from a second serotype. In certain embodiments, the AAV virus particle comprises a pseudotyped AAV. “Pseudotyped” AAV refers to an AAV that contains capsid proteins from one serotype and a viral genome including 5′-3′ ITRs of a second serotype. Pseudotyped AAV would be expected to have cell surface binding properties of the serotype from which the capsid protein is derived and genetic properties consistent with the serotype from which the ITRs are derived.

The genomic sequence of AAV as well as AAV rep genes, and cap genes are known in the art, and can be found in the literature and in public database such as the GenBank database. Table 1 below shows some example sequences for AAV genomes or AAV capsid sequences, and more are reviewed in Bernard N F et al., VIROLOGY, volume 2, chapter 69 (4th ed., Lippincott-Raven Publishers); Gao et al., (2004) J. Virol. 78:6381-6388; Naso M F et al., BioDrugs. 2017; 31(4): 317-334.

TABLE 1

| | GenBank |
|---|---|
| AAV | Accession No. |
| AAV1 | NC_002077; AF063497 |
| AAV2 | NC_001401 |
| AAV3 | NC_001729 |
| AAV4 | NC_001829 |
| AAV5 | Y18065, AF085716 |
| AAV6 | NC_001862 |
| AAV7 | NC_006260.1; AF513851 |
| AAV8 | NC_006261.1; AF513852, |
| Avian AAV ATCC | NC_004828 |
| VR-865 | |
| Avian AAV strain | NC_006263 |
| DA-1 | |
| Bovine AAV | NC_005889 |

Pharmaceutical Composition

In another aspect, the present disclosure provides a pharmaceutical composition comprising a virus particle as described above and a pharmaceutically acceptable carrier.

The term “pharmaceutically acceptable carrier” as used herein refers to any and all pharmaceutical carriers, such as solvents, dispersion media, coatings, antibacterial and antifungal agents, isotonic and absorption delaying agents, and the like that can facilitate storage and administration of the virus particles of the present disclosure to a subject. The pharmaceutically acceptable carriers can include any suitable components, such as without limitation, saline. Illustrative examples of saline include, without limitation, buffer saline, normal saline, phosphate buffer, citrate buffer, acetate buffer, bicarbonate buffer, sucrose solution, salts solution and polysorbate solution.

In certain embodiments, the pharmaceutical composition may further comprise additives, such as without limitation, stabilizers, preservatives, and transfection facilitating agents which assist in the cellular uptake of the medicines. Suitable stabilizers may include, without limitation, monosodium glutamate, glycine, EDTA and albumin (e.g. human serum albumin). Suitable preservatives may include, without limitation, 2-phenoxyethanol, sodium benzoate, potassium sorbate, methyl hydroxybenzoate, phenols, thimerosal, and antibiotics. Suitable transfection facilitating agents may include, without limitation, calcium ions.

The pharmaceutical composition may be suitable for administration via any suitable routes known in the art, including without limitation, parenteral, oral, enteral, buccal, nasal, topical, rectal, vaginal, transmucosal, epidermal, transdermal, dermal, ophthalmic, pulmonary, cardiac, subcutaneous, intraparenchymal, intracerebroventricular, or intrathecal administration routes.

The pharmaceutical composition can be administered to a subject in the form of formulations or preparations suitable for each administration route. Formulations suitable for administration of the pharmaceutical composition may include, without limitation, solutions, dispersions, emulsions, powders, suspensions, aerosols, sprays, nose drops, liposome based formulations, patches, implants and suppositories.

The formulations may conveniently be presented in unit dosage form and may be prepared by any methods well known in the art of pharmacy. Methods of preparing these formulations or compositions include the step of providing the exogenous nucleic acid of the present disclosure to one or more pharmaceutically acceptable carriers and, optionally, one or more adjuvants. Methods for making such formulations can be found in, for example, Remington's Pharmaceutical Sciences (Remington: The Science and Practice of Pharmacy, 19th ed., A. R. Gennaro (ed), Mack Publishing Co., N.J., 1995; R. Stribling et al., Proc. Natl. Acad. Sci. USA, 89:11277-11281 (1992); T. W. Kim et al., The Journal of Gene Medicine, 7(6): 749-758(2005); S. F. Jia et al., Clinical Cancer Research, 9:3462 (2003); A. Shahiwala et al., Recent patents on drug delivery and formulation, 1:1-9 (2007); A. Barnes et al., Current Opinion in Molecular Therapeutics 2000 2:87-93(2000), which references are incorporated herein by reference in their entirety).

Methods of Treatment

In another aspect, the present disclosure provides a method for treating a neurodegenerative disease in a subject using (e.g. by administering a therapeutically effective amount of) the TH variant, the pharmaceutical composition, the nucleotide construct, the vector (e.g. plasmid or viral vector), the cell, the virus particle, or the composition as described above.

In certain embodiments, the present disclosure provides a method of treating a neurodegenerative disease in a subject, comprising administering a therapeutically effective amount of the virus particles provided herein to the subject. The term “therapeutically effective amount” as used herein with respect to the virus particle, means that the amount of the virus particles delivered to the subject is sufficient to produce a therapeutic benefit in the subject, for example, to provide some alleviation, mitigation, or decrease in at least one clinical symptom in the subject. For example, a therapeutically effective amount of the exogenous nucleic acid can allow delivery into a sufficient number of the cells and expression of the TH variant (or derivative thereof) and AADC (or derivative thereof) in the subject to produce a therapeutically benefit. The therapeutic benefit can include for example, restoration of the motor symptoms of subjects with Parkinson's disease.

In certain embodiments, the therapeutically benefit of the viral particles, vectors, or compositions provided herein can be tested in a PD animal model. As used herein, the term “PD animal model” refers to an animal model capable of simulating critical phenotypes consistent with PD pathologies (e.g., neurodegeneration of dopaminergic neurons in the substantia nigra region of the brain). In one particular embodiment, the PD animal model used in the present disclosure is a mouse line called C57BL/6 whose dopaminergic neurons in the unilateral substantia nigra/ventral tegmental area (SNNTA) region are specifically killed by a toxic reagent (e.g., 6-hydroxydopamine, 6-OHDA). However, those skilled in the art should also understand that the establishment of a PD animal model provides guidance and methodology for the treatment of human PD. Consequently, a PD model of non-human primate that is evolutionarily closer to human in genetic relationship can theoretically help achieve the goal of clinical transformation. The mouse model used in this particular embodiment is only intended to illustrate that the enzyme composition of the present disclosure can improve PD dyskinesias and does not mean that it is only effective on mice. Those skilled in the art can reasonably expect that the enzyme composition of the present disclosure can improve PD dyskinesias of other species (e.g., human), based on the understanding of the prior art.

In certain embodiments, the virus particles provided herein are administered to the brain striatum of a subject.

As used herein, the term “subject” refers to any human or non-human animal. The term “non-human animal” refers to all vertebrates, such as mammals and non-mammals, such as non-human primates, sheep, dogs, cats, horses, cattle, chickens, rats, mice, amphibians and reptiles. Unless otherwise specified, the terms “patient” and “subject” are used interchangeably.

As used herein, the term “treating” or “method of treating” refers to both therapeutic and preventative measures. People in need of a treatment may include individuals already suffering from a specific disease or individuals who may eventually suffer from such disease.

In one embodiment, the virus particle comprising a nucleotide construct comprising the polynucleotide encoding a TH variant and an AADC is administered to the brain striatum of a subject for expression of the nucleotide of the TH variant and the AADC, which in turn causes ectopic synthesis of dopamine in the striatum, and eventually effectively restores the motor symptoms of subjects with Parkinson's disease.

In certain embodiments, the striatum is a caudate-putamen (CP) region.

In one embodiment, the present disclosure discloses use of the TH variant, the pharmaceutical composition, the nucleotide construct, the vector plasmid, the cell, the virus or the composition as described above in the manufacture of a medicament for treating a neurodegenerative disease in a subject.

In certain embodiments, the neurodegenerative disease is Parkinson's disease.

In certain embodiments, the subject is a mammal, preferably a human, a rat, or a mouse.

The Advantages of the Invention

The advantages of the present disclosure is that using the therapeutic method provided by the present disclosure, the enzyme composition for ectopic synthesis of dopamine can significantly increase the concentration of dopamine released by cells, which is significantly higher than other enzyme compositions. In addition, use of the AAV vector for delivering the above-mentioned exogenous genes results in effective expression of the nucleotide construct encoding the target enzyme composition in the striatum of the brain, thereby significantly improving the disease phenotype of PD. This indicates a great value of the enzyme composition with AAV as an expression vector of the present disclosure for application in gene therapy.

The experimental methods in the following examples are conventional unless otherwise specified.

### EXAMPLES

Methods and Materials

1. Construction of the AAV Vector Expressing the Enzyme Composition

The polynucleotide expressing the enzyme composition of the present disclosure and the AAV vector (Addgene: 26972) were digested with endonucleases BamHI and EcoRI for 1 hour at 37° C. to obtain the corresponding sticky ends. The target fragments purified by gel recovery were ligated with T4 DNA ligase overnight at 16° C. Mono-bacterial colonies were picked after transformation for cultivation, and vector plasmids were extracted and subjected to Sanger sequencing for sequence verification.

2. Culture and Transfection of 293 Cell Line In Vitro

The 293 cell line was cultured in DMEM supplemented with GlutaMAX and double antibiotics (penicillin and Streptomycin) at 37° C., 5% CO₂. Liposomal transfection (lipofectamine 3000 reagent) was performed when the density of 293 cells reached approximately 80% of the area of a 6-well plate. 293 cells in each well were transfected with 3 pg of the corresponding plasmid and continuously cultured for 48 hours for subsequent experiments.

3. High-Performance Liquid Chromatography (HPLC)

After the medium of 293 cells was sucked away, the cells were washed once with warm PBS, and then cultured in PBS (1 mL) for 1 hour. Lysates were harvested and then centrifuged at 3,000 rpm and 4° C. for 10 minutes. The supernatants were mixed with HClO₄ (0.6 M) at a ratio of 1:1, making the final concentration of HClO₄ 0.3M. Sufficiently mixed samples were centrifuged at 20,000 rpm for 15 minutes at 4° C. and the supernatants were applied to an HPLC system equipped with an ESA Coulochem III electrochemical detector (ESA Analytical). Catecholamines were separated using an Eclipse Plus C18 reversed phase column (3.5 μm, 2.1×150 mm) equilibrated with the flow phase at a rate of 0.2 mL/min, followed by electrochemical detection and calculation of dopamine concentration by integrating the specific peak.

4. Stereotactic Injection

A PD mouse model was constructed by injecting 6-OHDA into unilateral substantia nigra/ventral tegmental area (SN/VTA) on the genetic background of C57BL/6 mouse. Stereotactic administrations were performed for 500 nL injections of 6-OHDA (8 mg/mL) in unilateral SN/VTA regions. As a toxic reagent, 6-OHDA would specifically kill dopaminergic neurons. 6-OHDA was slowly infused at a speed of 50 nL/min and delivered at AP-3.6, ML-0.5 and DV-4.3.

In an experiment to verify role of the enzyme composition of the present disclosure in rescuing motor asymmetry of PD model mice, the inventors injected viral particles of AAV serotype 9 (titer: 1.95×10¹³ vg/mL) enclosing the vector plasmids expressing the target dual-enzyme composition into the caudate-putamen (CP) of striatum. Virus vectors expressing GFP (titer: 7.78×10¹² vg/mL) were used as a control. Three suitable injection sites were selected based on the standard mouse brain atlas: (1) AP 0.5, ML −2.0 and DV −3.0; (2) AP 0.5, ML −2.0 and DV −3.6; and (3) AP −0.6, ML −2.7 and DV −3.3. The injection volume at each site was 500 nL, and the injection speed was 50 nL/min using an infusion pump.

5. Apomorphine Rotation Test

Before the test, apomorphine was administered subcutaneously at the neck of the PD mouse model with the injection dosage measured by bodyweight (10 mg/kg). Animals were placed in a 10 cm diameter cylinder for habituation and then allowed to perform rotation tests. The results are expressed as the net turns per minute of apomorphine-induced rotation contralateral to the 6-OHDA lesion, which were calculated by the difference between contralateral and ipsilateral rotation turns divided by recording time of 60 minutes.

6. Immunohistochemistry

Animals were perfused transcardially with 4% PFA in PBS. Isolated brains were fixed in 4% PFA for about a week, and then subsequently dehydrated with 15% and 30% sucrose solutions. Cryostats sectioning were used to obtain brain slices with a thickness of 40 μm, containing the brain regions to be analyzed (SN/VTA and CP). After washing in PBS, the brain slices were incubated in block buffer (5% BSA, 0.3% TritonX-100 in PBS) for 2 h at room temperature. Then the slices were incubated with primary antibodies (anti-TH) overnight at 4° C., followed by the incubation of secondary antibodies that were corresponding to the source of the primary antibodies and with fluorescent groups (absorption wavelength of 488 nm) for 2 h at room temperature. All images were captured on the Olympus VS120 high-throughput fluorescence imaging system.

7. Sequence information involved in the experiment

| Sequence | | |
|---|---|---|
| Numbering | Name | Sequence |
| SEQ ID NO: | Amino acid | MPTPDATTPQAKGFRRAVSELDAKQAEAIMVRGQGAPGPSLTGSPVVPGTAAPAASYTPTPRSPRFI |
| 1 | sequence of | GRRQSLIEDARKEREAAVAAAAAAVPSEPGDPLEAVAFEEKEGKAVLNLLFSPRATKPSALSRAVKV |
| | full-length TH | FETFEAKIHNLETRPAQRPRAGGPHLEYFVRLEVRRGDLAALLSGVRQVSEDVRSPAGPKVPWFPR |
| | | KVSELDKCHHLVTKFDPDLDLDHPGFSDQVYRQRRKLIAEIAFQYRHGDPIPRVEYTAEEIATVVKEVY |
| | | TTLKGLYATHACGEHLEAFALLERFSGYREDNIPQLEDVSRFLKERTGFQLRPVAGLLSARDFLASLA |
| | | FRVFQCTQYIRHASSPMHSPEPDCCHELLGHVPMLADRTFAQFSQDIGLASLGASDEEIEKLSTLYW |
| | | FTVEFGLCKQNGEVKAYGAGLLSSYGELLHCLSEEPEIRAFDPEAAAVQPYQDQTYQSVYFVSESFS |
| | | DAKDKLRSYASRIQRPFSVKFDPYTLAIDVLDSPQAVRRSLEGVQDELDTLAHALSAIG |
| SEQ ID NO: | Amino acid | PSEPGDPLEAVAFEEKEGKAVLNLLFSPRATKPSALSRAVKVFETFEAKIHNLETRPAQRPRAGGPHL |
| 2 | sequence of | EYFVRLEVRRGDLAALLSGVRQVSEDVRSPAGPKVPVVFPRKVSELDKCHHLVTKFDPDLDLDHPGF |
| | TH with 90 | SDQVYRQRRKLIAEIAFQYRHGDPIPRVEYTAEEIATWKEVYTTLKGLYATHACGEHLEAFALLERFS |
| | amino acids | GYREDNIPQLEDVSRFLKERTGFQLRPVAGLLSARDFLASLAFRVFQCTQYIRHASSPMHSPEPDCC |
| | deleted | HELLGHVPMLADRTFAQFSQDIGLASLGASDEEIEKLSTLYWFTVEFGLCKQNGEVKAYGAGLLSSY |
| | | GELLHCLSEEPEIRAFDPEAAAVQPYQDQTYQSVYFVSESFSDAKDKLRSYASRIQRPFSVKFDPYTL |
| | | AIDVLDSPQAVRRSLEGVQDELDTLAHALSAIG |
| SEQ ID NO: | Amino acid | MYPYDVPDYAYPYDVPDYAPSEPGDPLEAVAFEEKEGKAVLNLLFSPRATKPSALSRAVKVFETFEA |
| 3 | sequence of | KIHHLETRPAQRPRAGGPHLEYFVRLEVRRGDLAALLSGVRQVSEDVRSPAGPKVPVVFPRKVSELD |
| | TH with 90 | KCHHLVTKFDPDLDLDHPGFSDQVYRQRRKLIAEIAFQYRHGDPIPRVEYTAEEIATVVKEVYTTLKGL |
| | amino acids | YATHACGEHLEAFALLERFSGYREDNIPQLEDVSRFLKERTGFQLRPVAGLLSARDFLASLAFRVFQ |
| | deleted and | CTQYIRHASSPMHSPEPDCCHELLGHVPMLADRTFAQFSQDIGLASLGASDEEIEKLSTLYWFTVEF |
| | with HA tag | GLCKQNGEVKAYGAGLLSSYGELLHCLSEEPEIRAFDPEAAAVQPYQDQTYQSVYFVSESFSDAKD |
| | | KLRSYASRIQRPFSVKFDPYTLAIDVLDSPQAVRRSLEGVQDELDTLAHALSAIG |
| SEQ ID NO: | Full-length | MNASEFRRRGKEMVDYVANYMEGIEGRQVYPDVEPGYLRPLIPAAAPQEPDTFEDIINDVEKIIMPGV |
| | amino acid | THWHSPYFFAYFPTASSYPAMLADMLCGAIGCIGFSWAASPACTELETVMMDWLGKMLELPKAFLN |
| | sequence of | EKAGEGGGVIQGSASEATLVALLAARTKVIHRLQAASPELTQAAIMEKLVAYSSDQAHSSVERAGLIG |
| | naturally | GVKLKAIPSDGNFAMRASALQEALERDKAAGLIPFFMVATLGTTTCCSFDNLLEVGPICNKEDIWLHV |
| | occurring | DAAYAGSAFICPEFRHLLNGVEFADSFNFNPHKWLLVNFDCSAMWVKKRTDLTGAFRLDPTYLKHS |
| | AADC | HQDSGLITDYRHWQIPLGRRFRSLKMWFVFRMYGVKGLQAYIRKHVQLSHEFESLVRQDPRFEICVE |
| | Isoform 1 | VILGLVCFRLKGSNKVNEALLQRINSAKKIHLVPCHLRDKFVLRFAICSRTVESAHVQRAWEHIKELAA |
| | | DVLRAERE |
| SEQ ID NO: | Full-length | MNASEFRRRGKEMVDYVANYMEGIEGRQVYPDVEPGYLRPLIPAAAPQEPDTFEDIINDVEKIIMPGA |
| 5 | amino acid | ASPACTELETVMMDWLGKMLELPKAFLNEKAGEGGGVIQGSASEATLVALLAARTKVIHRLQAASPE |
| | sequence of | LTQAAIMEKLVAYSSDQAHSSVERAGLIGGVKLKAIPSDGNFAMRASALQEALERDKAAGLIPFFMVA |
| | naturally | TLGTTTCCSFDNLLEVGPICNKEDIWLHVDAAYAGSAFICPEFRHLLNGVEFADSFNFNPHKWLLVNF |
| | occurring | DCSAMWVKKRTDLTGAFRLDPTYLKHSHQDSGLITDYRHWQIPLGRRFRSLKMWFVFRMYGVKGL |
| | AADC | QAYIRKHVQLSHEFESLVRQDPRFEICVEVILGLVCFRLKGSNKVNEALLQRINSAKKIHLVPCHLRDK |
| | Isoform 2 | FVLRFAICSRTVESAHVQRAVVEHIKELAADVLRAERE |
| SEQ ID NO: | Full-length | MNASEFRRRGKEMVDYVANYMEGIEGRQVYPDVEPGYLRPLIPAAAPQEPDTFEDIINDVEKIIMPGV |
| 6 | amino acid | THWHSPYFFAYFPTASSYPAMLADMLCGAIGCIGFSWAASPACTELETVMMDWLGKMLELPKAFLN |
| | sequence of | EKAGEGGGVIQGSASEATLVALLAARTKVIHRLQAASPELTQAAIMEKLVAYSSDQMVATLGTTTCCS |
| | naturally | FDNLLEVGPICNKEDIWLHVDAAYAGSAFICPEFRHLLNGVEFADSFNFNPHKWLLVNFDCSAMWVK |
| | occurring | KRTDLTGAFRLDPTYLKHSHQDSGLITDYRHWQIPLGRRFRSLKMWFVFRMYGVKGLQAYIRKHVQ |
| | AADC | LSHEFESLVRQDPRFEICVEVILGLVCFRLKGSNKVNEALLQRINSAKKIHLVPCHLRDKFVLRFAICSR |
| | Isoform 3 | TVESAHVQRAVVEHIKELAADVLRAERE |
| SEQ ID NO: | Full-length | |
| 7 | amino acid | MNASEFRRRGKEMVDYVANYMEGIEGRQVYPDVEPGYLRPLIPAAAPQEPDTFEDIINDVEKIIMPGG |
| | sequence of | SASEATLVALLAARTKVIHRLQAASPELTQAAIMEKLVAYSSDQAHSSVERAGLIGGVKLKAIPSDGNF |
| | naturally | AMRASALQEALERDKAAGLIPFFMVATLGTTTCCSFDNLLEVGPICNKEDIWLHVDAAYAGSAFICPE |
| | occurring | FRHLLNGVEFADSFNFNPHKVVLLVNFDCSAMVVVKKRTDLTGAFRLDPTYLKHSHQDSGLITDYRHW |
| | AADC | QIPLGRRFRSLKMWFVFRMYGVKGLQAYIRKHVQLSHEFESLVRQDPRFEICVEVILGLVCFRLKGS |
| | Isoform 4 | NKVNEALLQRINSAKKIHLVPCHLRDKFVLRFAICSRTVESAHVQRAVVEHIKELAADVLRAERE |
| SEQ ID NO: | Full-length | MNASEFRRRGKEMVDYVANYMEGIEGRQVYPDVEPGYLRPLIPAAAPQEPDTFEDIINDVEKIIMPGV |
| 8 | amino acid | THWHSPYFFAYFPTASSYPAMLADMLCGAIGCIGFSWAASPACTELETVMMDWLGKMLELPKAFLN |
| | sequence of | EKAGEGGGVIQMVATLGTTTCCSFDNLLEVGPICNKEDIWLHVDAAYAGSAFICPEFRHLLNGVEFAD |
| | naturally | SFNFNPHKWLLVNFDCSAMVVVKKRTDLTGAFRLDPTYLKHSHQDSGLITDYRHWQIPLGRRFRSLK |
| | occurring | MWFVFRMYGVKGLQAYIRKHVQLSHEFESLVRQDPRFEICVEVILGLVCFRLKGSNKVNEALLQRINS |
| | AADC | AKKIHLVPCHLRDKFVLRFAICSRTVESAHVQRAWEHIKELAADVLRAERE |
| | Isoform 5 | |
| SEQ ID NO: | Full-length | MNASEFRRRGKEMVDYVANYMEGIEGRQVYPDVEPGYLRPLIPAAAPQEPDTFEDIINDVEKIIMPGV |
| 9 | amino acid | THWHSPYFFAYFPTASSYPAMLADMLCGAIGCIGFSWAASPACTELETVMMDWLGKMLELPKAFLN |
| | sequence of | EKAGEGGGVIQGSASEATLVALLAARTKVIHRLQAASPELTQAAIMEKLVAYSSDQAHSSVERAGLIG |
| | naturally | GVKLKAIPSDGNFAMRASALQEALERDKAAGLIPFFMVATLGTTTCCSFDNLLEVGPICNKEDIWLHV |
| | occurring | DAAYAGSAFICPEFRHLLNGVEFADSFNFNPHKWLLVNFDCSAMWSRQPVRMLRLKKTCLVSAVVR |
| | AADC | RS |
| | Isoform 6 | |
| SEQ ID NO: | Full-length | NASEFRRRGKEMVDYVANYMEGIEGRQVYPDVEPGYLRPLIPAAAPQEPDTFEDIINDVEKIIMPGVT |
| 10 | amino acid | HWHSPYFFAYFPTASSYPAMLADMLCGAIGCIGFSWAASPACTELETVMMDWLGKMLELPKAFLNE |
| | sequence of | KAGEGGGVIQGSASEATLVALLAARTKVIHRLQAASPELTQAAIMEKLVAYSSDQAHSSVERAGLIGG |
| | AADC with | VKLKAIPSDGNFAMRASALQEALERDKAAGLIPFFMVATLGTTTCCSFDNLLEVGPICNKEDIWLHVD |
| | Myc tag | AAYAGSAFICPEFRHLLNGVEFADSFNFNPHKWLLVNFDCSAMVVVKKRTDLTGAFRLDPTYLKHSH |
| | attached at | QDSGLITDYRHWQIPLGRRFRSLKMVVFVFRMYGVKGLQAYIRKHVQLSHEFESLVRQDPRFEICVEV |
| | C terminus | ILGLVCFRLKGSNKVNEALLQRINSAKKIHLVPCHLRDKFVLRFAICSRTVESAHVQRAWEHIKELAAD |
| | | VLRAEREEQKLISEEDL |
| SEQ ID NO: | Full-length | atgcccacccccgacgccaccacgccacaggccaagggcttccgcagggccgtgtctgagctggacgcc |
| 11 | DNA | aagcaggcagaggccatcatggtaagagggcagggcgccccggggcccagcctcacaggctctccgtgg |
| | sequence of | cctggaactgcagccccagctgcatcctacacccccaccccaaggtccccgcggttcattgggcgcagg |
| | TH | cagagcctcatcgaggacgcccgcaaggagcgggaggcggcggtggcagcagcggccgctgcagtcccc |
| | | tcggagcccggggaccccctggaggctgtggcctttgaggagaaggaggggaaggccgtgctaaacctg |
| | | ctcttctccccgagggccaccaagccctcggcgctgtcccgagctgtgaaggtgtttgagacgtttgaa |
| | | gccaaaatccaccatctagagacccggcccgcccagaggccgcgagctgggggcccccacctggagtac |
| | | ttcgtgcgcctcgaggtgcgccgaggggacctggccgccctgctcagtggtgtgcgccaggtgtcagag |
| | | gacgtgcgcagccccgcggggcccaaggtcccctggttcccaagaaaagtgtcagagctggacaagtgt |
| | | catcacctggtcaccaagttcgaccctgacctggacttggaccacccgggcttctcggaccaggtgtac |
| | | cgccagcgcaggaagctgattgctgagatcgccttccagtacaggcacggcgacccgattccccgtgtg |
| | | gagtacaccgccgaggagattgccacctggaaggaggtctacaccacgctgaagggcctctacgccacg |
| | | cacgcctgcggggagcacctggaggcctttgctttgctggagcgcttcagcggctaccgggaagacaat |
| | | atcccccagctggaggacgtctcccgcttcctgaaggagcgcacgggcttccagctgcggcctgtggcc |
| | | ggcctgctgtccgcccgggacttcctggccagcctggccttccgcgtgttccagtgcacccagtatatc |
| | | cgccacgcgtcctcgcccatgcactcccctgagccggactgctgccacgagctgctggggcacgtgccc |
| | | atgctggccgaccgcaccttcgcgcagttctcgcaggacattggcctggcgtccctgggggcctcggat |
| | | gaggaaattgagaagctgtccacgctgtactggttcacggtggagttcgggctgtgtaagcagaacggg |
| | | gaggtgaaggcctatggtgccgggctgctgtcctcctacggggagctcctgcactgcctgtctgaggag |
| | | cctgagattcgggccttcgaccctgaggctgcggccgtgcagccctaccaagaccagacgtaccagtca |
| | | gtctacttcgtgtctgagagcttcagtgacgccaaggacaagctcaggagctatgcctcacgcatccag |
| | | cgccccttctccgtgaagttcgacccgtacacgctggccatcgacgtgctggacagcccccaggccgtg |
| | | cggcgctccctggagggtgtccaggatgagctggacacccttgcccatgcgctgagtgccattggctag |
| SEQ ID NO: | DNA | ccctcggagcccggggaccccctggaggctgtggcctttgaggagaaggaggggaaggccgtgctaaac |
| 12 | sequence of | ctgctcttctccccgagggccaccaagccctcggcgctgtcccgagctgtgaaggtgtttgagacgttt |
| | TH with 90 | gaagccaaaatccaccatctagagacccggcccgcccagaggccgcgagctgggggcccccacctggag |
| | amino acids | tacttcgtgcgcctcgaggtgcgccgaggggacctggccgccctgctcagtggtgtgcgccaggtgtca |
| | deleted | gaggacgtgcgcagccccgcggggcccaaggtcccctggttcccaagaaaagtgtcagagctggacaag |
| | | tgtcatcacctggtcaccaagttcgaccctgacctggacttggaccacccgggcttctcggaccaggtg |
| | | taccgccagcgcaggaagctgattgctgagatcgccttccagtacaggcacggcgacccgattccccgt |
| | | gtggagtacaccgccgaggagattgccacctggaaggaggtctacaccacgctgaagggcctctacgcc |
| | | acgcacgcctgcggggagcacctggaggcctttgctttgctggagcgcttcagcggctaccgggaagac |
| | | aatatcccccagctggaggacgtctcccgcttcctgaaggagcgcacgggcttccagctgcggcctgtg |
| | | gccggcctgctgtccgcccgggacttcctggccagcctggccttccgcgtgttccagtgcacccagtat |
| | | atccgccacgcgtcctcgcccatgcactcccctgagccggactgctgccacgagctgctggggcacgtg |
| | | cccatgctggccgaccgcaccttcgcgcagttctcgcaggacattggcctggcgtccctgggggcctcg |
| | | gatgaggaaattgagaagctgtccacgctgtactggttcacggtggagttcgggctgtgtaagcagaac |
| | | ggggaggtgaaggcctatggtgccgggctgctgtcctcctacggggagctcctgcactgcctgtctgag |
| | | gagcctgagattcgggccttcgaccctgaggctgcggccgtgcagccctaccaagaccagacgtaccag |
| | | tcagtctacttcgtgtctgagagcttcagtgacgccaaggacaagctcaggagctatgcctcacgcatc |
| | | cagcgccccttctccgtgaagttcgacccgtacacgctggccatcgacgtgctggacagcccccaggcc |
| | | gtgcggcgctccctggagggtgtccaggatgagctggacacccttgcccatgcgctgagtgccattggc |
| SEQ ID NO: | DNA | atgtacccatacgatgttccagattacgcttacccatacgatgttccagattacgctccctcggagccc |
| 13 | sequence of | ggggaccccctggaggctgtggcctttgaggagaaggaggggaaggccgtgctaaacctgctcttctcc |
| | TH with 90 | ccgagggccaccaagccctcggcgctgtcccgagctgtgaaggtgtttgagacgtttgaagccaaaatc |
| | amino acids | caccatctagagacccggcccgcccagaggccgcgagctgggggcccccacctggagtacttcgtgcgc |
| | deleted and | ctcgaggtgcgccgaggggacctggccgccctgctcagtggtgtgcgccaggtgtcagaggacgtgcgc |
| | with HA tag | agccccgcggggcccaaggtcccctggttcccaagaaaagtgtcagagctggacaagtgtcatcacctg |
| | | gtcaccaagttcgaccctgacctggacttggaccacccgggcttctcggaccaggtgtaccgccagcgc |
| | | aggaagctgattgctgagatcgccttccagtacaggcacggcgacccgattccccgtgtggagtacacc |
| | | gccgaggagattgccacctggaaggaggtctacaccacgctgaagggcctctacgccacgcacgcctgc |
| | | ggggagcacctggaggcctttgctttgctggagcgcttcagcggctaccgggaagacaatatcccccag |
| | | ctggaggacgtctcccgcttcctgaaggagcgcacgggcttccagctgcggcctgtggccggcctgctg |
| | | tccgcccgggacttcctggccagcctggccttccgcgtgttccagtgcacccagtatatccgccacgcg |
| | | tcctcgcccatgcactcccctgagccggactgctgccacgagctgctggggcacgtgcccatgctggcc |
| | | gaccgcaccttcgcgcagttctcgcaggacattggcctggcgtccctgggggcctcggatgaggaaatt |
| | | gagaagctgtccacgctgtactggttcacggtggagttcgggctgtgtaagcagaacggggaggtgaag |
| | | gcctatggtgccgggctgctgtcctcctacggggagctcctgcactgcctgtctgaggagcctgagatt |
| | | cgggccttcgaccctgaggctgcggccgtgcagccctaccaagaccagacgtaccagtcagtctacttc |
| | | gtgtctgagagcttcagtgacgccaaggacaagctcaggagctatgcctcacgcatccagcgccccttc |
| | | tccgtgaagttcgacccgtacacgctggccatcgacgtgctggacagcccccaggccgtgcggcgctcc |
| | | ctggagggtgtccaggatgagctggacacccttgcccatgcgctgagtgccattggc |
| SEQ ID NO: | Full-length | aacgcaagtgagtttcgaaggagagggaaggagatggtggattacgtggccaactacatggaaggcatt |
| 14 | DNA | gagggacgccaggtctaccctgacgtggagcccgggtacctgcggccgctgatccctgccgctgcccct |
| | sequence of | caggagccagacacgtttgaggacatcatcaacgacgttgagaagataatcatgcctggggtgacgcac |
| | naturally | tggcacagcccctacttcttcgcctacttccccactgccagctcgtacccggccatgcttgcggacatg |
| | occurring | ctgtgcggggccattggctgcatcggcttctcctgggcggcaagcccagcatgcacagagctggagact |
| | AADC | gtgatgatggactggctcgggaagatgctggaactaccaaaggcatttttgaatgagaaagctggagaa |
| | Isoform 1 | gggggaggagtgatccagggaagtgccagtgaagccaccctggtggccctgctggccgctcggaccaaa |
| | (synonymous | gtgatccatcggctgcaggcagcgtccccagagctcacacaggccgctatcatggagaagctggtggct |
| | replacement | tactcatccgatcaggcacactcctcagtggaaagagctgggttaattggtggagtgaaattaaaagcc |
| | of three | atcccctcagatggcaacttcgccatgcgtgcgtctgccctgcaggaagccctggagagagacaaagcg |
| | bases that | gctggcctgattcctttctttatggttgccaccctggggaccacaacatgctgctcctttgacaatctc |
| | changes the | ttagaagtcggtcctatctgcaacaaggaagacatatggctgcacgttgatgcagcctacgcaggcagt |
| | restriction | gcattcatctgccctgagttccggcaccttctgaatggagtggagtttgcagattcattcaactttaat |
| | site in the | ccccacaaatggctattggtgaattttgactgttctgccatgtgggtgaaaaagagaacagacttaacg |
| | DNA | ggagcctttagactggaccccacttacctgaagcacagccatcaggattcagggcttatcactgactac |
| | sequence) | cggcattggcagataccactgggcagaagatttcgctctttgaaaatgtggtttgtatttaggatgtat |
| | | ggagtcaaaggactgcaggcttatatccgcaagcatgtccagctgtcccatgagtttgagtcactggtg |
| | | cgccaggacccccgctttgaaatctgtgtggaagtcattctggggcttgtctgctttcggctaaagggt |
| | | tccaacaaagtgaatgaagctcttctgcaaagaataaacagtgccaaaaaaatccacttggttccatgt |
| | | cacctcagggacaagtttgtcctgcgctttgccatctgttctcgcacggtggaatctgcccatgtgcag |
| | | cgggcctgggaacacatcaaagagctggcggccgacgtgctgcgagcagagagggag |
| SEQ ID NO: | Full-length | atgaacgcaagtgaattccgaaggagagggaaggagatggtggattacgtggccaactacatggaaggc |
| 15 | DNA | attgagggacgccaggtctaccctgacgtggagcccgggtacctgcggccgctgatccctgccgctgcc |
| | sequence of | cctcaggagccagacacgtttgaggacatcatcaacgacgttgagaagataatcatgcctggggtgacg |
| | naturally | cactggcacagcccctacttcttcgcctacttccccactgccagctcgtacccggccatgcttgcggac |
| | occurring | atgctgtgcggggccattggctgcatcggcttctcctgggcggcaagcccagcatgcacagagctggag |
| | AADC | actgtgatgatggactggctcgggaagatgctggaactaccaaaggcatttttgaatgagaaagctgga |
| | Isoform 1 | gaagggggaggagtgatccagggaagtgccagtgaagccaccctggtggccctgctggccgctcggacc |
| | | aaagtgatccatcggctgcaggcagcgtccccagagctcacacaggccgctatcatggagaagctggtg |
| | | gcttactcatccgatcaggcacactcctcagtggaaagagctgggttaattggtggagtgaaattaaaa |
| | | gccatcccctcagatggcaacttcgccatgcgtgcgtctgccctgcaggaagccctggagagagacaaa |
| | | gcggctggcctgattcctttctttatggttgccaccctggggaccacaacatgctgctcctttgacaat |
| | | ctcttagaagtcggtcctatctgcaacaaggaagacatatggctgcacgttgatgcagcctacgcaggc |
| | | agtgcattcatctgccctgagttccggcaccttctgaatggagtggagtttgcagattcattcaacttt |
| | | aatccccacaaatggctattggtgaattttgactgttctgccatgtgggtgaaaaagagaacagactta |
| | | acgggagcctttagactggaccccacttacctgaagcacagccatcaggattcagggcttatcactgac |
| | | taccggcattggcagataccactgggcagaagatttcgctctttgaaaatgtggtttgtatttaggatg |
| | | tatggagtcaaaggactgcaggcttatatccgcaagcatgtccagctgtcccatgagtttgagtcactg |
| | | gtgcgccaggatccccgctttgaaatctgtgtggaagtcattctggggcttgtctgctttcggctaaag |
| | | ggttccaacaaagtgaatgaagctcttctgcaaagaataaacagtgccaaaaaaatccacttggttcca |
| | | tgtcacctcagggacaagtttgtcctgcgctttgccatctgttctcgcacggtggaatctgcccatgtg |
| | | cagcgggcctgggaacacatcaaagagctggcggccgacgtgctgcgagcagagagggagtag |
| SEQ ID NO: | Full-length | atgaacgcaagtgaattccgaaggagagggaaggagatggtggattacgtggccaactacatggaaggc |
| 16 | DNA | attgagggacgccaggtctaccctgacgtggagcccgggtacctgcggccgctgatccctgccgctgcc |
| | sequence of | cctcaggagccagacacgtttgaggacatcatcaacgacgttgagaagataatcatgcctggggcggca |
| | naturally | agcccagcatgcacagagctggagactgtgatgatggactggctcgggaagatgctggaactaccaaag |
| | occurring | gcatttttgaatgagaaagctggagaagggggaggagtgatccagggaagtgccagtgaagccaccctg |
| | AADC | gtggccctgctggccgctcggaccaaagtgatccatcggctgcaggcagcgtccccagagctcacacag |
| | Isoform 2 | gccgctatcatggagaagctggtggcttactcatccgatcaggcacactcctcagtggaaagagctggg |
| | | ttaattggtggagtgaaattaaaagccatcccctcagatggcaacttcgccatgcgtgcgtctgccctg |
| | | caggaagccctggagagagacaaagcggctggcctgattcctttctttatggttgccaccctggggacc |
| | | acaacatgctgctcctttgacaatctcttagaagtcggtcctatctgcaacaaggaagacatatggctg |
| | | cacgttgatgcagcctacgcaggcagtgcattcatctgccctgagttccggcaccttctgaatggagtg |
| | | gagtttgcagattcattcaactttaatccccacaaatggctattggtgaattttgactgttctgccatg |
| | | tgggtgaaaaagagaacagacttaacgggagcctttagactggaccccacttacctgaagcacagccat |
| | | caggattcagggcttatcactgactaccggcattggcagataccactgggcagaagatttcgctctttg |
| | | aaaatgtggtttgtatttaggatgtatggagtcaaaggactgcaggcttatatccgcaagcatgtccag |
| | | ctgtcccatgagtttgagtcactggtgcgccaggatccccgctttgaaatctgtgtggaagtcattctg |
| | | gggcttgtctgctttcggctaaagggttccaacaaagtgaatgaagctcttctgcaaagaataaacagt |
| | | gccaaaaaaatccacttggttccatgtcacctcagggacaagtttgtcctgcgctttgccatctgttct |
| | | cgcacggtggaatctgcccatgtgcagcgggcctgggaacacatcaaagagctggcggccgacgtgctg |
| | | cgagcagagagggagtag |
| SEQ ID NO: | Full-length | atgaacgcaagtgaattccgaaggagagggaaggagatggtggattacgtggccaactacatggaaggc |
| 17 | DNA | attgagggacgccaggtctaccctgacgtggagcccgggtacctgcggccgctgatccctgccgctgcc |
| | sequence of | cctcaggagccagacacgtttgaggacatcatcaacgacgttgagaagataatcatgcctggggtgacg |
| | naturally | cactggcacagcccctacttcttcgcctacttccccactgccagctcgtacccggccatgcttgcggac |
| | occurring | atgctgtgcggggccattggctgcatcggcttctcctgggcggcaagcccagcatgcacagagctggag |
| | AADC | actgtgatgatggactggctcgggaagatgctggaactaccaaaggcatttttgaatgagaaagctgga |
| | Isoform 3 | gaagggggaggagtgatccagggaagtgccagtgaagccaccctggtggccctgctggccgctcggacc |
| | | aaagtgatccatcggctgcaggcagcgtccccagagctcacacaggccgctatcatggagaagctggtg |
| | | gcttactcatccgatcagatggttgccaccctggggaccacaacatgctgctcctttgacaatctctta |
| | | gaagtcggtcctatctgcaacaaggaagacatatggctgcacgttgatgcagcctacgcaggcagtgca |
| | | ttcatctgccctgagttccggcaccttctgaatggagtggagtttgcagattcattcaactttaatccc |
| | | cacaaatggctattggtgaattttgactgttctgccatgtgggtgaaaaagagaacagacttaacggga |
| | | gcctttagactggaccccacttacctgaagcacagccatcaggattcagggcttatcactgactaccgg |
| | | cattggcagataccactgggcagaagatttcgctctttgaaaatgtggtttgtatttaggatgtatgga |
| | | gtcaaaggactgcaggcttatatccgcaagcatgtccagctgtcccatgagtttgagtcactggtgcgc |
| | | caggatccccgctttgaaatctgtgtggaagtcattctggggcttgtctgctttcggctaaagggttcc |
| | | aacaaagtgaatgaagctcttctgcaaagaataaacagtgccaaaaaaatccacttggttccatgtcac |
| | | ctcagggacaagtttgtcctgcgctttgccatctgttctcgcacggtggaatctgcccatgtgcagcgg |
| | | gcctgggaacacatcaaagagctggcggccgacgtgctgcgagcagagagggagtag |
| SEQ ID NO: | Full-length | atgaacgcaagtgaattccgaaggagagggaaggagatggtggattacgtggccaactacatggaaggc |
| 18 | DNA | attgagggacgccaggtctaccctgacgtggagcccgggtacctgcggccgctgatccctgccgctgcc |
| | sequence of | cctcaggagccagacacgtttgaggacatcatcaacgacgttgagaagataatcatgcctgggggaagt |
| | naturally | gccagtgaagccaccctggtggccctgctggccgctcggaccaaagtgatccatcggctgcaggcagcg |
| | occurring | tccccagagctcacacaggccgctatcatggagaagctggtggcttactcatccgatcaggcacactcc |
| | AADC | tcagtggaaagagctgggttaattggtggagtgaaattaaaagccatcccctcagatggcaacttcgcc |
| | Isoform 4 | atgcgtgcgtctgccctgcaggaagccctggagagagacaaagcggctggcctgattcctttctttatg |
| | | gttgccaccctggggaccacaacatgctgctcctttgacaatctcttagaagtcggtcctatctgcaac |
| | | aaggaagacatatggctgcacgttgatgcagcctacgcaggcagtgcattcatctgccctgagttccgg |
| | | caccttctgaatggagtggagtttgcagattcattcaactttaatccccacaaatggctattggtgaat |
| | | tttgactgttctgccatgtgggtgaaaaagagaacagacttaacgggagcctttagactggaccccact |
| | | tacctgaagcacagccatcaggattcagggcttatcactgactaccggcattggcagataccactgggc |
| | | agaagatttcgctctttgaaaatgtggtttgtatttaggatgtatggagtcaaaggactgcaggcttat |
| | | atccgcaagcatgtccagctgtcccatgagtttgagtcactggtgcgccaggatccccgctttgaaatc |
| | | tgtgtggaagtcattctggggcttgtctgctttcggctaaagggttccaacaaagtgaatgaagctctt |
| | | ctgcaaagaataaacagtgccaaaaaaatccacttggttccatgtcacctcagggacaagtttgtcctg |
| | | cgctttgccatctgttctcgcacggtggaatctgcccatgtgcagcgggcctgggaacacatcaaagag |
| | | ctggcggccgacgtgctgcgagcagagagggagtag |
| SEQ ID NO: | Full-length | atgaacgcaagtgaattccgaaggagagggaaggagatggtggattacgtggccaactacatggaaggc |
| 19 | DNA | attgagggacgccaggtctaccctgacgtggagcccgggtacctgcggccgctgatccctgccgctgcc |
| | sequence of | cctcaggagccagacacgtttgaggacatcatcaacgacgttgagaagataatcatgcctggggtgacg |
| | naturally | cactggcacagcccctacttcttcgcctacttccccactgccagctcgtacccggccatgcttgcggac |
| | occurring | atgctgtgcggggccattggctgcatcggcttctcctgggcggcaagcccagcatgcacagagctggag |
| | AADC | actgtgatgatggactggctcgggaagatgctggaactaccaaaggcatttttgaatgagaaagctgga |
| | Isoform 5 | gaagggggaggagtgatccagatggttgccaccctggggaccacaacatgctgctcctttgacaatctc |
| | | ttagaagtcggtcctatctgcaacaaggaagacatatggctgcacgttgatgcagcctacgcaggcagt |
| | | gcattcatctgccctgagttccggcaccttctgaatggagtggagtttgcagattcattcaactttaat |
| | | ccccacaaatggctattggtgaattttgactgttctgccatgtgggtgaaaaagagaacagacttaacg |
| | | ggagcctttagactggaccccacttacctgaagcacagccatcaggattcagggcttatcactgactac |
| | | cggcattggcagataccactgggcagaagatttcgctctttgaaaatgtggtttgtatttaggatgtat |
| | | ggagtcaaaggactgcaggcttatatccgcaagcatgtccagctgtcccatgagtttgagtcactggtg |
| | | cgccaggatccccgctttgaaatctgtgtggaagtcattctggggcttgtctgctttcggctaaagggt |
| | | tccaacaaagtgaatgaagctcttctgcaaagaataaacagtgccaaaaaaatccacttggttccatgt |
| | | cacctcagggacaagtttgtcctgcgctttgccatctgttctcgcacggtggaatctgcccatgtgcag |
| | | cgggcctgggaacacatcaaagagctggcggccgacgtgctgcgagcagagagggagtag |
| SEQ ID NO: | Full-length | atgaacgcaagtgaattccgaaggagagggaaggagatggtggattacgtggccaactacatggaaggc |
| 20 | DNA | attgagggacgccaggtctaccctgacgtggagcccgggtacctgcggccgctgatccctgccgctgcc |
| | sequence of | cctcaggagccagacacgtttgaggacatcatcaacgacgttgagaagataatcatgcctggggtgacg |
| | naturally | cactggcacagcccctacttcttcgcctacttccccactgccagctcgtacccggccatgcttgcggac |
| | occurring | atgctgtgcggggccattggctgcatcggcttctcctgggcggcaagcccagcatgcacagagctggag |
| | AADC | actgtgatgatggactggctcgggaagatgctggaactaccaaaggcatttttgaatgagaaagctgga |
| | Isoform 6 | gaagggggaggagtgatccagggaagtgccagtgaagccaccctggtggccctgctggccgctcggacc |
| | | aaagtgatccatcggctgcaggcagcgtccccagagctcacacaggccgctatcatggagaagctggtg |
| | | gcttactcatccgatcaggcacactcctcagtggaaagagctgggttaattggtggagtgaaattaaaa |
| | | gccatcccctcagatggcaacttcgccatgcgtgcgtctgccctgcaggaagccctggagagagacaaa |
| | | gcggctggcctgattcctttctttatggttgccaccctggggaccacaacatgctgctcctttgacaat |
| | | ctcttagaagtcggtcctatctgcaacaaggaagacatatggctgcacgttgatgcagcctacgcaggc |
| | | agtgcattcatctgccctgagttccggcaccttctgaatggagtggagtttgcagattcattcaacttt |
| | | aatccccacaaatggctattggtgaattttgactgttctgccatgtggtccagacaaccagtgcgtatg |
| | | ttaaggctgaagaaaacctgcttagtcagtgcggtggtgagaaggagttga |
| SEQ ID NO: | Full-length | aacgcaagtgagtttcgaaggagagggaaggagatggtggattacgtggccaactacatggaaggcatt |
| 21 | DNA | gagggacgccaggtctaccctgacgtggagcccgggtacctgcggccgctgatccctgccgctgcccct |
| | sequence of | caggagccagacacgtttgaggacatcatcaacgacgttgagaagataatcatgcctggggtgacgcac |
| | AADC with | tggcacagcccctacttcttcgcctacttccccactgccagctcgtacccggccatgcttgcggacatg |
| | Myc tag | ctgtgcggggccattggctgcatcggcttctcctgggcggcaagcccagcatgcacagagctggagact |
| | attached at | gtgatgatggactggctcgggaagatgctggaactaccaaaggcatttttgaatgagaaagctggagaa |
| | C terminus | gggggaggagtgatccagggaagtgccagtgaagccaccctggtggccctgctggccgctcggaccaaa |
| | | gtgatccatcggctgcaggcagcgtccccagagctcacacaggccgctatcatggagaagctggtggct |
| | | tactcatccgatcaggcacactcctcagtggaaagagctgggttaattggtggagtgaaattaaaagcc |
| | | atcccctcagatggcaacttcgccatgcgtgcgtctgccctgcaggaagccctggagagagacaaagcg |
| | | gctggcctgattcctttctttatggttgccaccctggggaccacaacatgctgctcctttgacaatctc |
| | | ttagaagtcggtcctatctgcaacaaggaagacatatggctgcacgttgatgcagcctacgcaggcagt |
| | | gcattcatctgccctgagttccggcaccttctgaatggagtggagtttgcagattcattcaactttaat |
| | | ccccacaaatggctattggtgaattttgactgttctgccatgtgggtgaaaaagagaacagacttaacg |
| | | ggagcctttagactggaccccacttacctgaagcacagccatcaggattcagggcttatcactgactac |
| | | cggcattggcagataccactgggcagaagatttcgctctttgaaaatgtggtttgtatttaggatgtat |
| | | ggagtcaaaggactgcaggcttatatccgcaagcatgtccagctgtcccatgagtttgagtcactggtg |
| | | cgccaggacccccgctttgaaatctgtgtggaagtcattctggggcttgtctgctttcggctaaagggt |
| | | tccaacaaagtgaatgaagctcttctgcaaagaataaacagtgccaaaaaaatccacttggttccatgt |
| | | cacctcagggacaagtttgtcctgcgctttgccatctgttctcgcacggtggaatctgcccatgtgcag |
| | | cgggcctgggaacacatcaaagagctggcggccgacgtgctgcgagcagagagggaggaacaaaaactc |
| | | atctcagaagaggatctg |
| SEQ ID NO: | Amino acid | YPYDVPDYA |
| 22 | sequence of | |
| | HA tag | |
| SEQ ID NO: | DNA | tacccatacgatgttccagattacgct |
| 23 | sequence of | |
| | HA tag | |
| SEQ ID NO: | Amino acid | EQKLISEEDL |
| 24 | sequence of | |
| | Myc tag | |
| SEQ ID NO: | DNA | gaacaaaaactcatctcagaagaggatctg |
| 25 | sequence of | |
| | Myc tag | |
| SEQ ID NO: | Amino acid | DYKDDDDK |
| 26 | sequence of | |
| | Flag tag | |
| SEQ ID NO: | DNA | gactacaaggacgatgatgacaag |
| 27 | sequence of | |
| | Flag tag | |
| SEQ ID NO: | DNA | gagggcagaggaagtctgctaacatgeggtgacgtcgaggagaatcctggccca |
| 28 | sequence of | |
| | T2A | |

### Example 1. Construction of an AAV Vector Expressing the Dual-Enzyme Composition

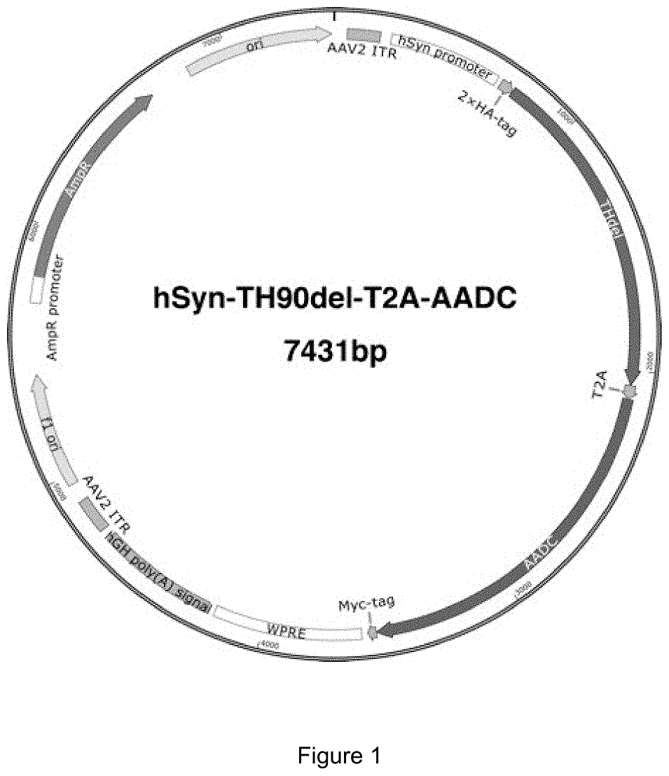

As shown in FIG. 1, an recombinant AAV vector expressing the dual-enzyme composition comprising the TH variant with a deletion of 90 amino acids at N terminus and a full-length AADC was constructed. The expression of downstream genes was regulated by the synapsin promoter. The polynucleotide expressing this dual-enzyme composition comprises three portions as shown below (from the 5′ to the 3′):

1). A polynucleotide encoding an N-terminally HA-tagged TH with 90 amino acid deleted at N terminus, which is set forth in SEQ ID NO: 13;

2) a T2A nucleotide sequence that encodes a self-cleaving peptide and is set forth in SEQ ID NO: 28; and

3) A polynucleotide encoding a C-terminally Myc-tagged full-length AADC, which is set forth in SEQ ID NO: 21.

The polynucleotide expressing the enzyme composition of the present disclosure was digested by endonucleases BamHI and EcoRI and subcloned to an AAV vector (Addgene: 26972).

As a control, the inventors simultaneously constructed an AAV vector carrying the synapsin promoter to induce GFP expression.

For better expression of the target sequences in the 293 cell line, so as to conveniently compare the capability of de novo dopamine synthesis among a series of compositions, each of which comprises a TH with a certain number of amino acids deleted at N terminus and a full-length AADC in the 293 cell line, the inventors simultaneously constructed a group of vector plasmids, with ubiquitin as a promoter, each of which expresses a composition comprising a full-length TH and a full-length AADC, a composition comprising another isomer of TH and a full-length AADC, a composition comprising a TH with 40 amino acids deleted at N terminus (i.e. amino acid residue 41-528 of SEQ ID NO:1) and a full-length AADC (SEQ ID NO: 4), a composition comprising a TH with 60 amino acids deleted at N terminus (i.e. amino acid residue 61-528 of SEQ ID NO:1) and a full-length AADC, a composition comprising a TH with 80 amino acids deleted at N terminus (i.e. amino acid residue 81-528 of SEQ ID NO:1) and a full-length AADC, a composition comprising a TH with 90 amino acids deleted at N terminus (i.e. amino acid residue 91-528 of SEQ ID NO:1, or SEQ ID NO: 2) and a full-length AADC, a composition comprising a TH with 100 amino acids deleted at N terminus (i.e. amino acid residue 101-528 of SEQ ID NO:1) and a full-length AADC, a composition comprising a TH with 120 amino acids deleted at N terminus (i.e. amino acid residue 121-528 of SEQ ID NO:1) and a full-length AADC, a composition comprising a TH with 150 amino acids deleted at N terminus (i.e. amino acid residue 151-528 of SEQ ID NO:1) and a full-length AADC, a composition comprising a TH with 164 amino acids deleted at N terminus (i.e. amino acid residue 165-528 of SEQ ID NO:1) and a full-length AADC, or a composition comprising a TH with 190 amino acids deleted at N terminus (i.e. amino acid residue 191-528 of SEQ ID NO:1) and a full-length AADC. The series of THs with N-terminal amino acid deletions were all attached with a HA tag at N terminus, and C terminus of the full-length AADC was attached with a Myc tag. Viral vectors expressing GFP with ubiquitin as a promoter were constructed as a control.

### Example 2. Functional Verification of the Enzyme Composition in Cultured Cell Lines In Vitro

To find the most efficient dual-enzyme composition for dopamine de novo synthesis, the inventors transfected the vector plasmids encoding a series of dual-enzyme compositions comprising a TH with amino acid deletions at N terminus and a full-length AADC as described above, respectively, into the 293 cell line with liposomes (lipofectamine 3000 reagent). As a negative control, the GFP expression vector was also transfected into the 293 cell line. After the incubation of the cultured cells in 37° C., 5% CO₂ for 48 hours, the cell culture medium was changed by PBS. After 1 hour of incubation in PBS, supernatant PBS and cell samples were harvested respectively.

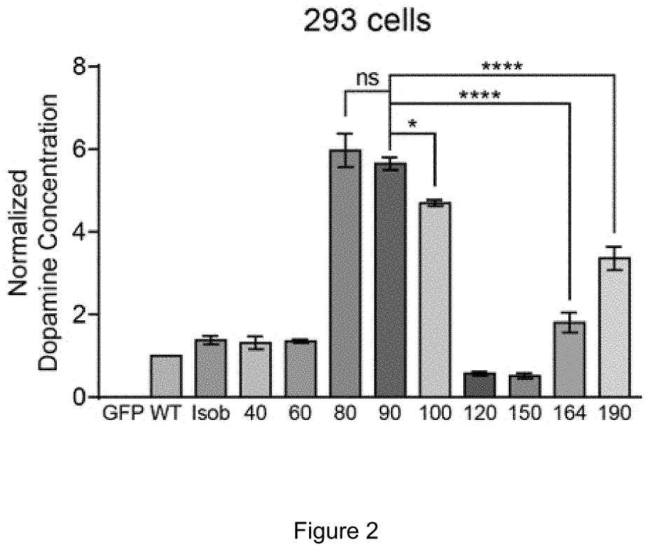

High-performance liquid chromatography (HPLC) was performed to detect the concentration of dopamine in the PBS samples above, i.e., the concentration of dopamine secreted by 293 cells. The results showed that dopamine was detected in all samples harvested from 293 cells expressing a series of dual-enzyme compositions comprising a TH with amino acid deletions at N terminus and a full-length AADC, but not in the samples expressing GFP (FIG. 2). This suggests that although the 293 cell line itself cannot synthesize and secrete dopamine, when the functional TH and AADC are introduced at the same time, the cells begin to synthesize and secrete dopamine. This proves that the various dual-enzyme compositions designed by the inventors can function normally, i.e., catalyze the de novo synthesis of dopamine.

The results further indicated that the dopamine concentration in the samples from 293 cells expressing the composition (90) comprising a TH with 90 amino acids deleted at N terminus and a full-length AADC was significantly higher than any of the samples from 293 cells expressing a composition (WT) comprising a full-length TH and a full-length AADC, a composition (Isob) comprising another isomer of TH and a full-length AADC, a composition (40) comprising a TH with 40 amino acids deleted at N terminus and a full-length AADC, a composition (60) comprising a TH with 60 amino acids deleted at N terminus and a full-length AADC, a composition (100) comprising a TH with 100 amino acids deleted at N terminus and a full-length AADC, a composition (120) comprising a TH with 120 amino acids deleted at N terminus and a full-length AADC, a composition (150) comprising a TH with 150 amino acids deleted at N terminus and a full-length AADC, a composition (164) comprising a TH with 164 amino acids deleted at N terminus and a full-length AADC, and a composition (190) comprising a TH with 190 amino acids deleted at N terminus and a full-length AADC. But the difference was not significant when compared to that in the 293 cell sample expressing the composition (80) comprising a TH with 80 amino acids deleted at N terminus and a full-length AADC (see FIG. 2). These data confirmed that an increase in dopamine concentration will negatively regulate the activity of TH, thereby limiting its ability to synthesize dopamine ectopically, and this dilemma can be solved by using a version of constitutively activated TH with certain amino acid residues deleted at N terminus. More importantly, the inventors found the optimal type of constitutively activated TH with certain amino acid deletions at N terminus through comparison, i.e., the TH with 80 or 90 amino acids deleted at N terminus. The dual-enzyme composition provided by the present disclosure is a composition comprising a TH with 80 or 90 amino acids deleted at N terminus and a full-length AADC. The results indicate that this dual-enzyme composition has better ability of de novo dopamine synthesis than that of other types of compositions comprising a TH with certain deletions at N terminus and a full-length AADC. In summary, the dual-enzyme composition provided by the present disclosure can function best de novo dopamine synthesis. While it has been known that the TH with certain deletion at N terminus is in a constitutively activated state, the present disclosure provides the optimal type of the constitutively activated TH variant.

### Example 3. The Construction of PD Model Mice

The 8-week-old C57BL/6 mouse line was selected to construct a PD model. According to the standard mouse brain atlas, a stereotactic injection of 500 nL 6-OHDA (8 mg/mL) into the unilateral SNNTA region was performed. 6-OHDA is a toxic drug that specifically kills dopaminergic neurons. Two weeks later, apomorphine was injected subcutaneously at the neck of the mice with the injection dosage measured by bodyweight (10 mg/kg), and a rotation test was then performed. Mice with phenotype of apomorphine-induced motor asymmetry which presented rotation contralateral to the 6-OHDA lesion were selected for subsequent experiments.

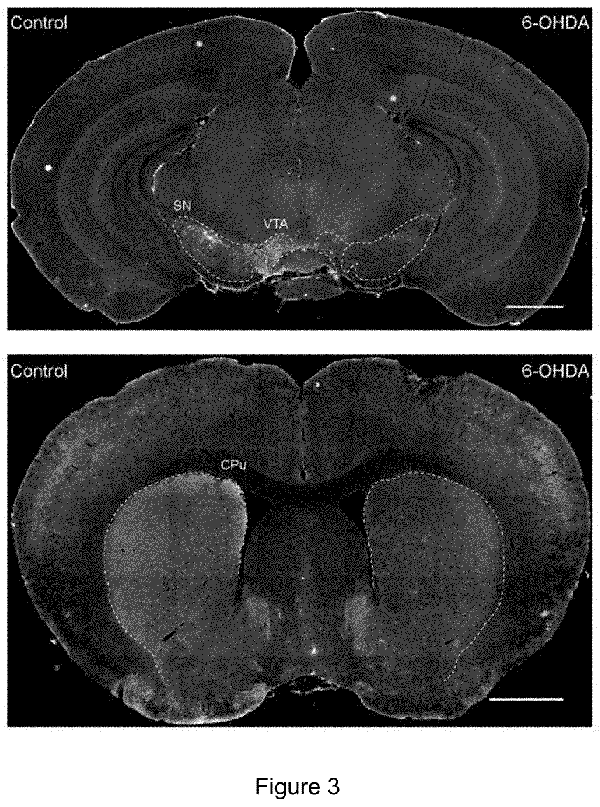

The immunohistochemical assays of the cryostats brain slices from the mice with motor asymmetry were carried out, which showed that TH-positive staining signals were detected both in SNNTA and striatal CP regions contralateral to the 6-OHDA lesions as controls in PD mice, but not in regions ipsilateral to the lesions (see FIG. 3). This result showed that 6-OHDA caused effective lesion to dopaminergic neurons in SNNTA projecting to CP.

In summary, the PD mouse model was successfully constructed for subsequent rescue experiments.

### Example 4. Phenotype Rescue of PD Mouse Model by the Dual-Enzyme Composition

The vector plasmid expressing the composition (TH90del/AADC) comprising a TH with 90 amino acids deleted at N terminus and a full-length AADC was packaged into viral particles of AAV serotype 9 (titer: 1.95×10¹³ vg/mL) for in vivo expression in PD mice. GFP-expressing plasmids were packaged into AAV particles (GFP, titer: 7.78×10¹² vg/mL) as controls.

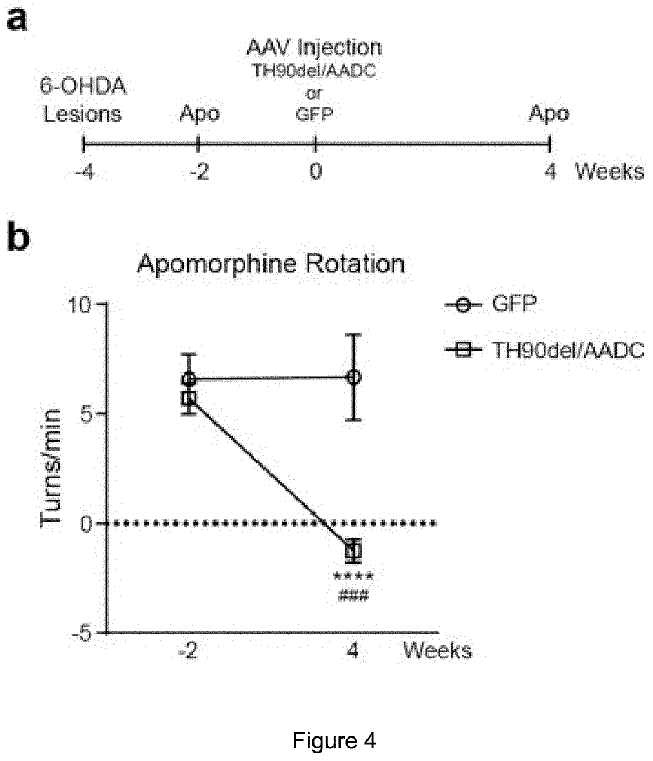

The PD mouse model successfully constructed in Example 3 was used to perform the phenotype rescue experiment according to the workflow shown in FIG. 4a. AAV packaging TH90del/AADC or GFP was intrastriatally injected with a stereotaxic apparatus in three appropriated injection sites that were selected based on the standard mouse brain atlas. Each site received 500 nL viral injection. Four weeks after the viral administrations, apomorphine-induced rotational tests were performed by subcutaneous apomorphine injections at neck, whose dosages were measured by bodyweight (10 mg/kg). The rescue effectiveness was indicated as the decrease in net turns per minute of apomorphine-induced rotation contralateral to the 6-OHDA lesion, which were calculated by the difference between contralateral and ipsilateral rotation turns divided by recording time of 60 minutes. The results showed significantly decreased net turns per minute of contralateral rotation of PD animals that received injections of TH90del/AADC viral vectors in apomorphine-induced motor asymmetry tests 4 weeks after viral administrations, comparing to those before viral injections and those of the control group (GFP) (FIG. 4b). Taken together, it has been demonstrated that the particular dual-enzyme composition (i.e., TH90del/AADC) of the present disclosure can effectively function in vivo and increase the dopamine concentration in striatal CP region which is innervated by dopaminergic neurons in SN, and thereby significantly ameliorating the apomorphine-induced motor asymmetry in PD mouse model. Therefore, the dual-enzyme composition (TH90del/AADC) provided by the present disclosure has potential therapeutic effects on PD.

Although the enzyme composition used in the embodiments and/or examples is from human, those skilled in the art should reasonably expect that the human or mouse dual-enzyme composition will have good therapeutic effects on mouse models or human clinical trials, since the protein homology between human and mouse TH or AADC is 83% or 89%, respectively, based on the disclosure of the present disclosure.

In summary, the inventors have illustrated the detailed description of the present disclosure, but the scope of which is beyond this description. Those skilled in the art should understand that the scope of the present disclosure includes varied and modified embodiments that should fall within the protection scope of the present disclosure.

1. A tyrosine hydroxylase variant comprising an amino acid sequence set forth in SEQ ID NO: 1 except for an N-terminal deletion of 60 to 120 amino acid residues, or a fragment, a derivative or an analog thereof having at least 80% sequence identity.

2. The tyrosine hydroxylase variant of claim 1, comprising an amino acid sequence set forth in SEQ ID NO: 1 except for an N-terminal deletion of 80 to 100 amino acid residues, or a fragment, a derivative or an analog thereof having at least 80% sequence identity.

3. The tyrosine hydroxylase variant of claim 2, comprising an amino acid sequence set forth in SEQ ID NO: 1 except for an N-terminal deletion of 80 to 90 amino acid residues, or a fragment, a derivative or an analog thereof having at least 80% sequence identity.

4. The tyrosine hydroxylase variant of claim 2, comprising an amino acid sequence set forth in SEQ ID NO: 2 or a fragment, a derivative or an analog thereof having at least 80% sequence identity.

5. The tyrosine hydroxylase variant of claim 4, further comprising a tag protein attached to N terminus or C terminus.

6. The tyrosine hydroxylase variant of claim 5, wherein the tag protein is HA, Myc or Flag.

7. The tyrosine hydroxylase variant of claim 1, comprising an amino acid sequence set forth in SEQ ID NO: 3.

8. A composition, comprising the tyrosine hydroxylase variant of claim 1.

9. The composition of claim 8, further comprising an aromatic L-amino acid decarboxylase.

10. The composition of claim 9, wherein the aromatic L-amino acid decarboxylase comprises an amino acid sequence set forth in any of SEQ ID NOs: 4-9 or a fragment, a derivative or an analog thereof having at least 80% sequence identity.

11. The composition of claim 10, wherein the aromatic L-amino acid decarboxylase further comprises a tag protein attached to the N terminus or the C terminus.

12. The composition of claim 11, wherein the tag protein is HA, Myc or Flag.

13. The composition of claim 8, wherein the aromatic L-amino acid decarboxylase has an amino acid sequence set forth in SEQ ID NO: 10.

14. A polynucleotide construct, comprising a first polynucleotide encoding the tyrosine hydroxylase variant of claim 1, and optionally a second polynucleotide encoding the aromatic L-amino acid decarboxylase.

15. The polynucleotide construct of claim 14, wherein the first polynucleotide has a nucleotide sequence set forth in SEQ ID NO: 12 or 13, or has a nucleotide sequence having at least 80% sequence identity to SEQ ID NO: 12 or 13.

16. The polynucleotide construct of claim 14, wherein the second polynucleotide has a nucleotide sequence set forth in any of SEQ ID NOs: 14-21, or has a nucleotide sequence having at least 80% sequence identity to any of SEQ ID NOs: 14-21.

17. The polynucleotide construct of claim 14, further comprising a promoter operably linked to the first polynucleotide and/or to the second polynucleotide.

18. The polynucleotide construct of claim 17, wherein the promoter comprises a neuron-specific promoter.

19. A vector, comprising the polynucleotide construct of claim 14.

20. The vector of claim 19, wherein the first polynucleotide and the second polynucleotide are constructed in one vector, or in different vectors.

21. The vector of claim 20, wherein the first polynucleotide and the second polynucleotide are constructed in one vector, and the vector further comprises a third polynucleotide inserted between the first polynucleotide and the second polynucleotide.

22. The vector of claim 21, wherein the third polynucleotide encodes for a self-cleavable sequence and/or an internal ribosome entry site (IRES).

23. The vector of claim 19, wherein the vector is selected from the group consisting of simplex virus vector, adenovirus vector, and adeno-associated virus vector.

24. A host cell comprising or transfected by the vector of claim 19.

25. A virus comprising a virus genome, wherein the virus genome comprises the polynucleotide construct of claim 14 or comprises a nucleic acid expressed from the polynucleotide construct of claim 14.

26. A pharmaceutical composition, comprising the virus of claim 25 and a pharmaceutically acceptable carrier.

27. (canceled)

28. (canceled)

29. (canceled)

30. A method of treating a neurodegenerative disease in a subject in need thereof, comprising administering to the subject a therapeutically effective amount of the vector of claim 19.

31. The method of claim 30, wherein the neurodegenerative disease is Parkinson's disease.

32. The method of claim 30, wherein the subject is a mammal, preferably a human, a rat, or a mouse.

33. A method of treating a neurodegenerative disease in a subject in need thereof, comprising administering to the subject a therapeutically effective amount of the virus of claim 25.

34. The method of claim 33, wherein the neurodegenerative disease is Parkinson's disease.

35. The method of claim 33, wherein the subject is a mammal, preferably a human, a rat, or a mouse.


SEQUENCE LISTING (28 sequences)

SEQ ID NO: 1 - protein, 528 aa, Homo sapiens
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 2 - protein, 437 aa, Artificial Sequence
  Amino acid sequence of TH with a deletion of 90 amino acids
PSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 3 - protein, 456 aa, Artificial Sequence
  Amino acid sequence of TH with an HA tag and a deletion of 90 amino acids
MYPYDVPDYAYPYDVPDYAPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 4 - protein, 480 aa, Artificial Sequence
  Naturally-occurring amino acid sequence of full-length AADC Isoform 1
MNASEFRRRGKEMVDYVANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 5 - protein, 442 aa, Artificial Sequence
  Naturally-occurring amino acid sequence of full-length AADC Isoform 2
MNASEFRRRGKEMVDYVANYMEGIEGRQVYPDVEPGYLRPLIPAAAPQEPDTFEDIINDVEKIIMPGAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 6 - protein, 432 aa, Artificial Sequence
  Naturally-occurring amino acid sequence of full-length AADC Isoform 3
MNASEFRRRGKEMVDYVANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 7 - protein, 402 aa, Artificial Sequence
  Naturally-occurring amino acid sequence of full-length AADC Isoform 4
MNASEFRRRGKEMVDYVANYMEGIEGRQVYPDVEPGYLRPLIPAAAPQEPDTFEDIINDVEKIIMPGGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 8 - protein, 387 aa, Artificial Sequence
  Naturally-occurring amino acid sequence of full-length AADC Isoform 5
MNASEFRRRGKEMVDYVANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 9 - protein, 338 aa, Artificial Sequence
  Naturally-occurring amino acid sequence of full-length AADC Isoform 6
MNASEFRRRGKEMVDYVANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWSRQPVRMLRLKKTCLVSAVVRRS

SEQ ID NO: 10 - protein, 489 aa, Artificial Sequence
  Amino acid sequence of full-lenght AADC with myc tag attached to C terminus
NASEFRRRGKEMVDYVANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAEREEQKLISEEDL

SEQ ID NO: 11 - DNA, 1587 bp, Homo sapiens
atgcccacccccgacgccaccacgccacaggccaagggcttccgcagggccgtgtctgagctggacgccaagcaggcagaggccatcatggtaagagggcagggcgccccggggcccagcctcacaggctctccgtggcctggaactgcagccccagctgcatcctacacccccaccccaaggtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 12 - DNA, 1311 bp, Artificial Sequence
  DNA sequence encoding TH with a deletion of 90 amino acid
ccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggc

SEQ ID NO: 13 - DNA, 1368 bp, Artificial Sequence
  DNA sequence encoding TH with an HA tag and a deletion of 90 amino acids
atgtacccatacgatgttccagattacgcttacccatacgatgttccagattacgctccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggc

SEQ ID NO: 14 - DNA, 1437 bp, Artificial Sequence
  Mutant DNA sequence encoding full-length AADC Isoform 1
aacgcaagtgagtttcgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggacccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggag

SEQ ID NO: 15 - DNA, 1443 bp, Artificial Sequence
  Naturally-occurring DNA sequence encoding full-length AADC Isoform 1
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 16 - DNA, 1329 bp, Artificial Sequence
  Naturally occurring DNA sequence encoding full-length AADC Isoform 2
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 17 - DNA, 1299 bp, Artificial Sequence
  Naturally occurring DNA sequence encoding full-length AADC Isoform 3
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcagatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 18 - DNA, 1209 bp, Artificial Sequence
  Natually-ocurring DNA sequence encoding full-length AADC Isoform 4
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctgggggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 19 - DNA, 1164 bp, Artificial Sequence
  Natually-ocurring DNA sequence encoding full-length AADC Isoform 5
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 20 - DNA, 1017 bp, Artificial Sequence
  Natually-ocurring DNA sequence encoding full-length AADC Isoform 6
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtggtccagacaaccagtgcgtatgttaaggctgaagaaaacctgcttagtcagtgcggtggtgagaaggagttga

SEQ ID NO: 21 - DNA, 1467 bp, Artificial Sequence
  DNA sequence encoding full-length AADC with myc tag attached to C terminus
aacgcaagtgagtttcgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggacccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggaggaacaaaaactcatctcagaagaggatctg

SEQ ID NO: 22 - protein, 9 aa, Artificial Sequence
  Amino acid sequence of HA tag
YPYDVPDYA

SEQ ID NO: 23 - DNA, 27 bp, Artificial Sequence
  DNA sequence encoding HA tag
tacccatacgatgttccagattacgct

SEQ ID NO: 24 - protein, 10 aa, Artificial Sequence
  Amino acid sequnce of Myc tag
EQKLISEEDL

SEQ ID NO: 25 - DNA, 30 bp, Artificial Sequence
  DNA sequence encoding Myc tag
gaacaaaaactcatctcagaagaggatctg

SEQ ID NO: 26 - protein, 8 aa, Artificial Sequence
  Amino acid sequence of Flag tag
DYKDDDDK

SEQ ID NO: 27 - DNA, 24 bp, Artificial Sequence
  DNA sequence encoding Flag tag
gactacaaggacgatgatgacaag

SEQ ID NO: 28 - DNA, 54 bp, Artificial Sequence
  DNA sequence encoding T2A
gagggcagaggaagtctgctaacatgcggtgacgtcgaggagaatcctggccca